(19) United States
(12) Patent Application Publication
(10) Pub. No.: US 2023/0107004 A1
(43) Pub. Date: Apr. 6, 2023

(54) REAGENTLESS ELECTROCHEMICAL BIOSENSOR

(71) Applicant: THE GOVERNING COUNCIL OF THE UNIVERSITY OF TORONTO, Toronto (CA)

(72) Inventors: Shana KELLEY, Toronto (CA); Edward SARGENT, Toronto (CA); Jagotamoy DAS, Mississauga (CA); Surath GOMIS, Toronto (CA); Jenise B. CHEN, Toronto (CA); Sharif AHMED, Scarborough (CA); Hanie YOUSEFI, Toronto (CA); Dingran CHANG, Toronto (CA); Alam MAHMUD, Toronto (CA)

(21) Appl. No.: 17/904,846

(22) PCT Filed: Mar. 2, 2021
(86) PCT No.: PCT/CA2021/050270

§ 371 Date: Aug. 23, 2022

Related U.S. Application Data
(60) Provisional application No. 62/984,080, filed on Mar. 2, 2020.
(60) Provisional application No. 63/035,338, filed on Jun. 5, 2020.

(51) Int. Cl. G01N 33/543; C12Q 1/70; G01N 27/327; G01N 33/566

(52) U.S. Cl. CPC G01N 33/5438; C12Q 1/701; G01N 27/3277; G01N 33/566; G01N 2333/165; G01N 2469/10; G01N 2469/20

(57) ABSTRACT

A biosensor comprising an electrode and inverted molecular pendulums (iMPs) is described. Each IMP includes a linker bound to the electrode, and an analyte receptor and a redox reporter both bound to the linker. The redox reporter is reactive at positive potential when the linker presents a net negative charge and reactive at negative potential when the linker presents a net positive charge. Upon application of an electric field, the biosensor is characterized by an iMPs unbound state, where no analyte is bound to the receptor, at which the iMPs are displaced towards the electrode and electron transfer from the iMPs towards the electrode occurs at an unbound electron transfer rate, and an iMPs bound state, where the analyte is bound to the receptor, at which the iMPs are displaced towards the electrode and electron transfer from the iMPs towards the electrode occurs at a bound electron transfer rate.

### RELATED PATENT APPLICATION

The present application claims priority from U.S. provisional patent application No. 62/984,080 filed Mar. 2, 2020, and U.S. provisional patent application No. 63/035,338 filed Jun. 5, 2020, the disclosures of which are hereby incorporated by reference in their entirety.

### TECHNICAL FIELD

The technical field generally relates to an electrochemical biosensor that can track molecular analytes, such as analytes present in biological fluids. More particularly, the technical field relates to a reagentless electrochemical biosensor, a method of use thereof and a disposable device comprising such biosensor, to detect molecular analytes.

### BACKGROUND

The ability to sense biological inputs using self-contained devices, without relying on external reagents or reporters, can open opportunities to collect information about human health and environment. Currently, a very limited set of molecular inputs can be detected using this type of sensor format. Indeed, electrochemical sensors for glucose, lactate and a handful of other molecules are the only examples that are compatible with the development of dynamic detection systems for monitoring in physiological systems. Existing reagentless electrochemical sensors compatible with physiological monitoring applications are generally based on DNA aptamers that serve as recognition elements. While powerful for the collection of pharmacokinetic data in living systems where analyte concentrations are high, aptamer-based sensors typically have low binding affinities that render them incompatible with many sensing applications. The development of versatile sensors that could track molecular analytes in biological fluids is desirable.

Moreover, existing diagnostic testing solutions that have been employed to combat the COVID-19 pandemic are slow and complex. The requirement for sample processing, amplification, and testing within centralized hospital laboratories has created bottlenecks and backlogs, limiting the utility of test results and widespread testing. The introduction of point-of-care tests that can be conducted without a requirement for laboratory facilities will allow testing outside of metropolitan areas, but will not provide a solution to scale testing volumes significantly given the low throughput of existing devices. The present inability to contain the spread of SARS-CoV-2 highlights the need for radically new testing approaches. There is a great need for fast screening tools that can be used to rapidly assess patients and individuals in the community. There is also a need for accurately testing modalities that are amenable to decentralized at-home testing. Similarly, these technologies are needed for testing of other infectious diseases, at-home or in-hospital monitoring of chronic diseases, and for personal wellness monitoring.

### SUMMARY

The present technology relates to an electrochemical biosensor useful for detecting various analytes present in fluids such as biological biofluids for instance.

According to one aspect, the electrochemical biosensor comprises a plurality of inverted molecular pendulums (iMPs) and a biosensor electrode, wherein each one of the iMPs comprises
a linker having a first end and a second end, the first end of the linker being bound to a surface of the biosensor electrode,
a receptor for a target analyte, the receptor being bound to the second end of the linker, and
a redox reporter bound to the linker, wherein the redox reporter is reactive at positive potential when the linker presents a net negative charge and the redox reporter is reactive at negative potential when the linker presents a net positive charge,

wherein upon application of an electric field, the biosensor is characterized by
an iMPs unbound state, where no target analyte is bound to the receptor, at which the iMPs are displaced towards the biosensor electrode surface and electron transfer from the iMPs towards the biosensor electrode occurs at an unbound electron transfer rate,
an iMPs bound state, where the target analyte is bound to the receptor, at which the iMPs are displaced towards the biosensor electrode surface and electron transfer from the iMPs towards the biosensor electrode occurs at a bound electron transfer rate.

In an optional embodiment, the biosensor is such that upon binding of the target analyte to the receptor at the applied electric field, an electrochemical signal is produced translating a difference between the unbound electron transfer rate and the bound electron transfer rate.

In another optional embodiment, the biosensor is such that upon application of the electric field, the redox reporter causes an electron transfer as the iMPs approach the biosensor electrode surface.

In another optional embodiment, the biosensor is such that the electron transfer rate is dependent on a time rate at which the iMPs are displaced.

In another optional embodiment, the biosensor is such that the unbound electron transfer rate is dependent on a time rate at which the unbound iMPs are displaced.

In another optional embodiment, the biosensor is such that the bound electron transfer rate is dependent on a time rate at which the bound iMPs are displaced.

In another optional embodiment, the biosensor is such that the iMPs displacement towards the biosensor electrode surface substantially corresponds to a tilting movement of the iMPs.

In another optional embodiment, the biosensor is such that upon application of the electric field, the redox reporter touches the biosensor electrode surface and the electron transfer is based on a redox reaction or electron tunneling current.

In another optional embodiment, the biosensor is such that the redox reporter is bound to the linker close to the second end thereof. In another optional embodiment, the redox reporter is covalently bound to the linker.

In another optional embodiment, the biosensor is such that the linker comprises a double-stranded DNA (dsDNA), single-stranded DNA (ssDNA), charged polymers, uncharged polymers, or any combination thereof.

In another optional embodiment, the biosensor is such that the linker comprises a dsDNA and has a length ranging from about 10mer to about 100mer.

In another optional embodiment, the biosensor is such that the linker comprises a ssDNA and has a length ranging from about 15mer to about 60mer.

In another optional embodiment, the linker is negatively charged and comprises a DNA/DNA duplex, a PNA/DNA duplex, a PNA/PNA duplex where one or both of the PNA are modified with negative charged amino acids, a rigid anionic polyelectrolyte, a rigid negatively charged peptide, or any combination thereof.

In another optional embodiment, the biosensor is such that the redox reporter comprises ferrocene, [Co(GA)₂(phen)] (GA=glycolic acid, phen=1,10-phenathroline), metal nanoparticles (e.g., Au, Pt, Pd, Ag, Cu), pyrroloquinoline quinone (PQQ), benzoquine, Osmium(III) complexes such as Os(bpy)Cl₂³⁺, diphenylamine, or any combination thereof.

In another optional embodiment, the biosensor is such that the redox reporter has a redox state change above 0 mV.

In another optional embodiment, the biosensor is such that the linker is positively charged and comprises a PNA/PNA duplex with lysines, a rigid cationic polyelectrolyte, a rigid positively charged peptide, or any combination thereof.

In another optional embodiment, the biosensor is such that the redox reporter comprises methylene blue, ruthenium(III) complexes such as Ru(NH₃)₆³⁺, neutral red, toluidine blue, phenosafranine, or any combination thereof.

In another optional embodiment, the biosensor is such that the redox reporter has a redox state change below 0 mV.

In another optional embodiment, the biosensor is such that the linker has a length ranging from about 5 nm to about 20 nm.

In another optional embodiment, the biosensor is such that the linker is rigid along a length thereof.

In another optional embodiment, the biosensor is such that the linker comprises a dsDNA having a first DNA strand and a second DNA strand, the first DNA strand is bound to the surface of the biosensor electrode at the first end of the linker and the second DNA strand is modified by removing nucleotides from the 3′ end thereof thereby defining an iMPs flexibility region at the first end of the linker. In another optional embodiment, the biosensor is such that the number of removed nucleotides is adjusted such that the difference between the number of nucleotides in the first DNA strand and the number of nucleotides in the second DNA strand is from 1 to 15.

In another optional embodiment, the biosensor is such that the iMPs are rigid in a rigid region comprised between the second end of the linker and the flexibility region.

In another optional embodiment, the biosensor is such that the iMPs form a molecular monolayer at the surface of the biosensor electrode.

In another optional embodiment, the biosensor is such that the receptor comprises an antibody, a nanobody, an antigen, an aptamer, an aptamer fragment, a molecular imprint, a protein receptor, DNA, a microorganism, a protein/enzyme substrate, or any combination thereof. In another optional embodiment, the receptor comprises an antibody, a protein or an aptamer.

In another optional embodiment, the biosensor is such that the receptor comprises an antibody selected from the group consisting of anti-MRSA antibody, anti-MSSA antibody, anti-E. coli antibody, anti-Tuberculosis antibody, anti-pseudomonas aeruginosa antibody, anti-S-protein antibody, anti-troponin I antibody, anti-troponin T antibody, anti-IgE antibody, anti-BNP antibody, anti-BDNF antibody, anti-p53 antibody, anti-AFP antibody, anti-CEA antibody, anti-TRX antibody, anti-IL-8 antibody, and anti-IL-6 antibody.

In another optional embodiment, the biosensor is such that the receptor comprises a protein selected from the group consisting of S-protein, Brain natriuretic peptide (BNP) protein, troponin I protein, troponin T protein, Natural Human IgE protein, Brain-derived neurotrophic factor (BDNF) protein, Thioredoxin (TRX), IL-6 protein, IL-8 protein, Carcino Embryonic Antigen (CEA) protein, alpha 1 Fetoprotein (AFP), and p53 protein.

In another optional embodiment, the biosensor is such that the receptor comprises an aptamer binding the Receptor binding domain (RBD) site of an S-protein or a BNP-specific aptamer.

In another optional embodiment, the biosensor is such that the biosensor electrode comprises a glassy carbon electrode, a carbon nanotube-modified electrode, an indium tin oxide (ITO) electrode, a platinum electrode, a silver electrode, a gold electrode, or a palladium electrode. In another optional embodiment, the electrode comprises a gold nanostructured microelectrode or a gold wire electrode.

In another optional embodiment, the biosensor is such that target analyte is the SARS-CoV -2 virus, the linker comprises a double-stranded DNA (dsDNA), the receptor comprises a protein, an aptamer or an antibody specific to the SARS-CoV-2 spike protein and the redox reporter comprises ferrocene.

In another optional embodiment, the biosensor is such that the receptor comprises SARS1 polyclonal anti s1 protein antibody, SARS1 S1 protein, Receptor binding domain (RBD) binding IgG, SARS2 polyclonal anti s1 protein antibody, SARS2 Monoclonal anti s protein antibody, SARS2 Polyclonal anti S1 protein antibody, SARS2 S1 protein, or SARS2 S1 protein.

In another optional embodiment, the biosensor is such that the receptor comprises an aptamer targeting the Receptor-Binding Domain of the SARS-CoV-2 spike.

According to another aspect there is provided a method of detecting a target analyte in a sample comprising:
providing the electrochemical biosensor as defined herein;
contacting said biosensor with the sample; and
detecting the electrochemical signal, wherein detection of said signal indicates the presence of the target analyte.

According to another aspect there is provided a method for in situ detection of a target analyte in a biological fluid, comprising:
contacting the electrochemical biosensor as defined herein with the biological fluid; and
detecting the electrochemical signal, wherein detection of said signal indicates the presence of the target analyte.

According to another aspect there is provided the use of the electrochemical biosensor as defined herein to detect the presence of a target analyte in a sample.

According to another aspect there is provided the use of the electrochemical biosensor as defined herein to detect the presence of a target analyte in a biological fluid.

In one optional embodiment, the biological fluid is saliva, blood, urine, tears, sweat or faeces.

In one optional embodiment, the biosensor, the method or the use as defined herein, is such that the target analyte is a small molecule, a macromolecule, a prokaryotic or eukaryotic cell-derived component (e.g., nucleic acid material), a virus, a bacterium, an antibody, a protein, a cellular extract, or any combination thereof.

In one optional embodiment, the biosensor, the method or the use as defined herein, is such that the target analyte is a protein comprising Brain natriuretic peptide (BNP) protein, troponin I protein, troponin T protein, Natural Human IgE protein, Brain-derived neurotrophic factor (BDNF) protein, Thioredoxin (TRX), IL-6 protein, IL-8 protein, Carcino Embryonic Antigen (CEA) protein, alpha 1 Fetoprotein (AFP), p53 protein, or spike protein (S-protein), and the target analyte is different than the receptor.

In one optional embodiment, the biosensor, the method or the use as defined herein, is such that the target analyte is an antibody comprising an anti-S-protein antibody, anti-troponin I antibody, anti-troponin T antibody, Anti-IgE antibody, anti-BNP antibody, Anti-BDNF antibody, anti-p53 antibody, anti-AFP antibody, anti-CEA antibody, anti-TRX antibody, anti-IL-8 antibody, or anti-IL-6 antibody, and the target analyte is different than the receptor.

In one optional embodiment, the biosensor, the method or the use as defined herein, is such that the target analyte is a bacterium comprising Methicillin-resistant Staphylococcus aureus (MRSA), Methicillin-susceptible Staphylococcus aureus (MSSA), E. coli, Tuberculosis (TB) or Pseudomonas Aeruginosa.

In one optional embodiment, the biosensor, the method or the use as defined herein, is such that the target analyte is a coronavirus.

In one optional embodiment, the biosensor, the method or the use as defined herein, is such that the target analyte is a SARS-CoV or a MERS-CoV virus, such as the SARS-CoV-2 virus.

In one optional embodiment, the biosensor, the method or the use as defined herein, is such that the target analyte is an antibody that is specific to a coronavirus.

In one optional embodiment, the biosensor, the method or the use as defined herein, is such that the target analyte is an antibody that is specific to a SARS-CoV or a MERS-CoV virus, such as the SARS-CoV-2 virus.

According to another aspect there is provided a disposable device for detecting a target analyte in a sample comprising:
a collector for collecting the sample;
a sensing component comprising the biosensor as defined herein; and
a connector for connecting the sensing component to an electrochemical measurement device;
   wherein the collector and the sensing component are designed for allowing contact between the iMPs of the biosensor and the collected sample.

In one optional embodiment, the disposable device is such that the collector is designed to at least partially encase the sensing component.

In one optional embodiment, the disposable device is such that the collector comprises a first portion and a second portion, the first portion being able to contain the collected sample and the second portion encasing the sensing component. In one optional embodiment, the first portion comprises a nozzle and a sampling reservoir. In one optional embodiment, the nozzle comprises microfluidic channels optionally treated with a hydrophilic coating. In one optional embodiment, the disposable device is such that the nozzle comprises silica capillary tubes.

In one optional embodiment, the disposable device is such that the collector and the connector are provided with complementary locking means to secure the sensing component within the device.

In one optional embodiment, the disposable device is such that the sensing component further comprises two working electrodes, a counter electrode and a reference electrode. In one optional embodiment, the electrode of the biosensor and the two working electrodes comprise gold. In one optional embodiment, the counter electrode comprises platinum and the reference electrode comprises silver.

In one optional embodiment, the disposable device is such that each electrode is in the form of a wire.

In one optional embodiment, the disposable device is such that the sensing component has a cylindrical form and the wires are positioned spaced apart within the sensing component along a length thereof. In one optional embodiment, the electrodes are held in a matrix of a non-conductive material. In one optional embodiment, the non-conductive material comprises a silicon resin.

In one optional embodiment, the disposable device is such that the IMPs are bound to a first end of the biosensor electrode wire and the IMPs are exposed to the sample when the first end is in contact with the collected sample. In one optional embodiment, a first end of each electrode wire is in contact with the sample when collected in the collector. In one optional embodiment, a second end of the wires are in contact with the connector.

In one optional embodiment, the disposable device is such that the sample comprises a biological fluid which is saliva, blood, urine, tears, sweat or faeces.

In one optional embodiment, the disposable device is for targeting an analyte that is the SARS-CoV-2 virus.

Other objects, advantages and features of the present technology will become more apparent upon reading of the following non-restrictive description of specific embodiments and examples thereof, with reference to the accompanying drawings.

### BRIEF DESCRIPTION OF THE DRAWINGS

FIG. 1: Theoretical modeling the dynamics of an inverted molecular pendulum (iMP) tethered to an electrode surface. a) Model parameters. The dynamics of the iMP in the presence of an applied field were modeled by considering the drag force (F_{d}), the force exerted by the applied field (Fₑ), and electrostatic interactions of neighboring iMPs (F_{c}). The length of the linker separating the “bob” of the pendulum (L), the distance between neighboring iMPs (d), the average angle of the iMPs relative to the surface (θ), the diffusion coefficient (D), molecular charge (q), and the applied electric field (Eₐₚₚ) were all varied to explore iMP dynamics. Under an applied positive potential, a negatively charged iMP is attracted to the sensor surface. The transit time of the iMP would be reflected in the modulation of τ. b) Dependence of τ on the length of a negatively charged linker for an unbound iMP. c) Dependence of τ on the magnitude of the applied electric field for an unbound iMP. d) Dependence of τ on added molecular charge for an unbound iMP. e) Dependence of τ on increasing diffusion coefficient (equivalent to a bound analyte decreasing from a 3 nm to 0 nm radius) and modulations by analyte charge. f) Modeled current decays expected if the dynamics of the iMP could be monitored using an electrochemically active label in the absence and presence of a molecular cargo with an appreciable 10 nm radius and +10e charge, demonstrating how changes in τ can translate to measurable microampere current.

FIG. 2: Modulation of iMP dynamics by protein binding according to one embodiment. a) A protein-binding iMP was constructed using a DNA linker and an antibody specific for cardiac troponin I. A redox reporter was incorporated into the DNA linker that would be oxidized at an electrode potential compatible with the electric field required to transport the iMP to the surface. b) Observation of binding-induced modulation of iMP transport using chronoamperometry in the presence and absence of cardiac troponin I. c) Time-dependent current modulation for iMP in the absence and presence of cardiac troponin I. d) Dependence of iMP dynamics on modifying the fluid matrix. A secondary antibody to cardiac troponin I was used for the bound state. e) Comparison of observed and calculated τ values for varied drag-modulated fluid matrices. A secondary antibody to cardiac troponin I was used for the bound state. The data points are experimental measurements and the lines are the theoretical prediction.

FIG. 3: Panel of proteins and biofluids that can be monitored using iMPs. a) Concentration-dependent signal change for cardiac troponin I in buffered solution. The dotted line represents the signal (+3 times of the standard deviations) recorded with a control sensor. b) Construction and testing of a panel of iMPs specific for cardiac, inflammation, stress, and cancer markers. c) Detection of cardiac troponin I in different biofluids including saliva, sweat, tear, urine, and blood.

FIG. 4: iMP-based monitoring of a cardiac marker in living animals. a) Short-term cycling of iMP signals. By adding and diluting samples of cardiac troponin I, the sensor responds accordingly. b) Long-term incubation and response of iMP in saliva. The sensor does not deteriorate over time, producing data after 3 weeks of incubation in saliva. c) Measurement of cardiac troponin I in saliva both ex situ and in situ using the iMP. To induce cardiac dysfunction, mice were treated with doxorubicin for four days and saliva samples were collected at day 5 for ex situ measurements, in addition to single-point measurements were collected within the mouth. d) In situ continuous monitoring of cardiac troponin I in a murine model. Mice were injected with cardiac troponin I and the iMP signal change was monitored continuously after 10 minutes of sensor equilibration.

FIG. 5: Principles a wearable assay for real time electrochemical monitoring of disease. A) The biosensor functions by applying an electrode potential. Applied positive potential pulls the negatively charged probe to the sensor surface, which brings a redox-probe closer to the sensor surface. Addition of the target protein causes the reduction of electron transfer kinetics due to slower falling. Applied negative potential, on the other hand, pushes the probe away from the surface. B) The biosensor platform described enables a broad range of applications in personalized health monitoring. The platform is compatible with the analysis of proteins that are important physiological markers of stress, allergy, cardiovascular health, inflammation, and cancer. The sensing approach is compatible with making measurements in blood, saliva, urine, tears and sweat. C) The system can be integrated into a wearable device for real-time monitoring of disease.

FIG. 6: Direct Viral Particle Detection. a) Direct detection of pseudotyped SARS viral particle expressing spike protein on their surface membrane in human saliva. b) Negative control: pseudotyped viral particle without spike protein expressed on their membrane. c) Limit of detection of the sensor detecting SARS CoV spike protein diluted in human saliva. Protein concentrations as low as 0.1 pg/mL in 100 μL sample volumes can be detected with the sensor. d) The sensor detecting SARS CoV pseudotyped viral particles diluted to concentrations of 10⁴ particles/mL in 100 μL samples.

FIG. 7: Limit of Detection for Viral Particle Detection. The sensitivity of the system was evaluated for detection of viral particles (using RBD binding IgG as receptor). The system successfully detected 10⁴ particle/mL concentration of 100 μL viral particle sample.

FIG. 8: Decay curve simulations. Simulation of decay curves of the unbound, isolated spike protein, and SARS-CoV-2 detection, in the order of increasingly shallower slope. This indicates that the larger the bound analyte is, the shallower the current decay curve will be as larger bound analytes take longer to fall due to competing hydrodynamic drag against the electrostatic pull between the DNA linker and electrode. In this case, the SARS-CoV-2 virus is much larger in size (˜100 nm) compared to isolated S protein (<10 nm).

FIG. 9: Time dynamics—Antibody based detection of SARS CoV-2 spike-protein. The real-time detection of S protein (kinetics of binding event). A polyclonal antibody is used as the receptor and detection of S protein starts at 5 minutes of incubation.

FIG. 10: Aptamer-based sensor (CoV-2 S protein detection). Reconfiguration of the sensor from antibody to DNA based sensor. The receptor was changed to an RBD binding DNA probe (aptamer) and the efficiency of the system was evaluated.

FIG. 11: Time dynamics of aptamer-based detection of spike protein. The real-time detection of S protein (kinetics of binding event): RBD binding aptamer is used as the receptor and detection of S protein starts at 5 minutes of incubation.

FIG. 12: LOD for aptamer-spike protein sensing. The sensitivity of the new aptamer-based sensor was evaluated detecting spike protein in 0.1×PBS. The system can detect concentrations as low as 100 fg/mL in 100 μL samples.

FIG. 13: Detection of heat-treated spike protein. Heat treated spike protein (following standard heat treatment protocol used for inactivation of CoV-2 virus, 30 minutes at 65° C.) was used to evaluate the performance of the system. Heat-treatment does not have an adverse effect on systems detection capability.

FIG. 14: Density of iMPs on sensor to detect viral particles. Optimization of the sensors platform for detecting viral particles. Due to relatively large size of virus (in comparison to protein analytes), certain characteristics of the system can be optimized—such as the probe density on the surface of electrode. A range of dsDNA-based probe concentrations for surface modification of electrode were studied to measure the virus capturing efficiency. 0.1 μM of dsDNA-based probe showed best performance with viral particles. This is 10-fold lower compared to optimized concentrations for protein detection.

FIG. 15: Detection of SARS-CoV-2 in human COVID-19 patient samples. Detection of SARS-CoV-2 within 5 minutes, and signal differentiation between negative and positive patient samples (in blinded tests) was performed.

FIG. 16: In vivo animal tests of iMP sensors in COVID-19 infected and healthy hamsters. A handheld device prototype using iMP sensors was used to detect for SARS-CoV-2 in the mouth (saliva) of sedated hamsters, with distinct signals observed between healthy and infected animals.

FIG. 17: Detection of circulating antibodies. Reconfiguration of the sensor's receptor from an antibody to a protein. This sensor can detect a specific antibody in bodily fluids. The sensor was used to detect anti-S-protein antibodies (concentration: 1 ng/mL, 100 μL sample).

FIG. 18: Schematic representation of a handheld device comprising a disposable sensing device, for viral detection.

FIG. 19: Schematic representation of a handheld device comprising a disposable sensing device, for viral detection. a) possible electrode configuration within the device. b) the electrode wire bundle can be encased in a PDMS matrix/sheath to orient the electrode wires and expose only the tips for sample access.

FIG. 20: Long term stability of iMPs. iMP sensors were stored for 8 months at 4 degrees Celsius. Stored iMPs showed functionality after this period, with measurable signal with 10 and 1000 pg/m L of target.

FIG. 21: Bacteria detection using iMPs. Control bacteria were incubated with iMPs with anti-MRSA antibody as the receptor. a) Positive (MRSA) demonstrated an observable signal compared to the initial control trace; b) Negative (Pseudomonas aeruginosa) does not show a signal with overlapping initial and target traces.

FIG. 22: Analytical resolution of iMP sensors for BNP in buffer. Sensors designed to detect BNP protein were able to discern between multiple protein concentrations between 80 pg/mL to 1 ng/mL spiked in 0.1×PBS.

FIG. 23: Detection of BNP in different body fluids. BNP sensors were able to detect BNP in human saliva and whole human blood after 50 minutes target incubation.

FIG. 24: Analytical resolution of iMP sensors for BNP in whole human blood. Sensors designed to detect BNP protein were able to discern between multiple protein concentrations between 80 pg/mL to 1 ng/mL spiked in whole human blood.

FIG. 25: BNP detection using iMP aptamer-based sensor. iMP sensors were designed using BNP-specific aptamers as receptors, and detection was verified for a range of BNP concentrations from 100 pg/mL to 1 ng/mL. a) Collected chronoamperometric current signals using standard Epsilon device. b) Concentration-dependent signal change for BNP in buffered solution.

FIG. 26: Signal response from single-stranded DNA-based probes is dependent on the probe length. In a range of lengths, larger probes give smaller currents.

FIG. 27: Single-stranded DNA-based probes and double-stranded DNA-based probes performance. Single-stranded DNA-based probes produce sharper chronoamperometric peaks.

FIG. 28: Introducing flexibility into the iMP linker (by modifying the base pairing of the DNA). Flexibility can enhance current signals.

FIG. 29: Capacitive vs. faradaic current contributions in iMPs. Removing the redox reporter from the iMP demonstrates the small capacitive contributions of the system, indicating the prominent faradaic current from the redox molecule.

FIG. 30: iMP mechanism verification. An iMP with a dsDNA (negatively charge) linker was constructed with a methylene blue redox reporter (requiring a negative potential). No meaningful signal was produced, indicating electrostatic attraction requirements of the iMP system. System was tested at both a) negative potentials and b) positive potentials.

### DETAILED DESCRIPTION

There is provided a new electrochemical biosensor for detecting analytes in a sample such as biological fluids, which is reagentless (i.e., reagent-free), meaning that the biosensor does not require the use of external reagents to collect the information.

More particularly, the biosensor comprises a plurality of inverted molecular pendulums (iMPs) and an electrode. Each one of the iMPs comprises a linker, a receptor and a redox reporter. The linker has two extremities and is bound at one extremity to the surface of the electrode and to the receptor at the other extremity. The receptor is designed to receive/bind to a target analyte. The redox reporter is bound to the linker. When the redox reporter is reactive at positive potential, then the linker presents a net negative charge and when the redox reporter is reactive at negative potential, then the linker presents a net positive charge. Once an electric field is applied, the biosensor can be characterized by two different states: i) an iMPs unbound state, where no target analyte is bound to the receptor, at which the iMPs are displaced towards the electrode surface and electron transfer from the iMPs towards the electrode occurs at an unbound electron transfer rate; and ii) an iMPs bound state, where the target analyte is bound to the receptor, at which the iMPs are displaced towards the electrode surface and electron transfer from the iMPs towards the electrode occurs at a bound electron transfer rate.

Further details on the characteristics of the biosensor and its uses for detecting analytes will be provided below.

### ABBREVIATIONS

Below is provided the meaning of the abbreviations used in the present description.

### CA: Chronoamperometry

### Da: Dalton

DNA: Deoxyribonucleic acid
PNA: Peptide nucleic acid

### iMP: Inverted Molecular Pendulum

dsDNA: double-stranded DNA
ssDNA: single-stranded DNA
GA: glycolic acid
Phen: 1,10-phenathroline
PQQ: pyrroloquinoline quinone
PBS: Phosphate buffered saline
TCEP: tris(2-carboxyethyl)phosphine hydrochloride
MCH: 6-mercapto-1-hexanol
BSA: Bovine serum albumin
NME: nanostructured microelectrode

### CTI: Cardiac Troponin I

THC: tetrahydrocannabinol
CBD: cannabidiol
LOD: limit of detection
PDMS: polydimethylsiloxane
LCD: liquid crystal display
CE: control electrode
RE: reference electrode
WE: working electrode
DMSO: Dimethyl sulfoxide
MRSA: Methicillin-resistant Staphylococcus aureus
MSSA: Methicillin-susceptible Staphylococcus aureus

### TB: Tuberculosis

TBTA: tris((1-benzyl-4-triazolyl)methyl)amine
RBD: Receptor binding domain
BNP: Brain natriuretic peptide
BDNF: Brain-derived neurotrophic factor

### TRX: Thioredoxin

### CEA: Carcino Embryonic Antigen

AFP: alpha 1 Fetoprotein

### S-protein: Spike-protein.

### DEFINITIONS

The use of the word “a” or “an” when used in conjunction with the term “comprising” in the claims and/or the specification may mean “one” but it is also consistent with the meaning of “one or more”, “at least one”, and “one or more than one”.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “target analyte” refers to an analyte, such as a molecular analyte present in biological fluids. The target analyte comprises the entity to be detected using the present biosensor. More particularly, the target analyte can bind to the Inverted Molecular Pendulum via a receptor and its detection can be performed by an electrochemical signal, as will be explained in more detail below. In some embodiments, the target analyte can be small molecules, macromolecules, prokaryotic or eukaryotic cell-derived components such as nucleic acid material, proteins, viruses, bacteria, antibodies, or cellular extracts, or any combination thereof. In some embodiments, the target analyte can include a virus. The virus, although not limited to, can for example be a ribovirus (e.g., rotavirus, Japanese encephalitis virus, yellow fever virus, measles morbillivirus, Lassa mammarenavirus, hantaviruses, influenza viruses, coronaviruses, hepatitis B virus, and the human immunodeficiency viruses), adenovirus, filovirus (e.g., ebolavirus), herpesevirus, poxvirus, parvovirus, reovirus, picomavirus, togavirus, orthomyxovirus, rhabdovirus, retrovirus, hepadnavirus, lentivirus, norovirus. In one embodiment, the target analyte is a coronavirus. For instance, the target analyte can be a coronavirus such as a SARS-CoV or a MERS-CoV virus, e.g, the SARS-CoV-2 virus. In another embodiment, the target analyte can be an antibody that is specific to a virus, for instance a coronavirus. For instance, the target analyte can be an antibody that is specific to a SARS-CoV or a MERS-CoV virus, such as the SARS-CoV-2 virus. In some embodiments, the target analyte can include a bacterium. The bacterium, although not limited to, can for example be Methicillin-resistant Staphylococcus aureus (MRSA), Methicillin-susceptible Staphylococcus aureus (MSSA), E. coli, Tuberculosis (TB), Pseudomonas Aeruginosa. In some embodiments, the target analyte can include a protein selected from Brain natriuretic peptide (BNP) protein, troponin I protein, troponin T protein, Natural Human IgE protein, Brain-derived neurotrophic factor (BDNF) protein, Thioredoxin (TRX), IL-6 protein, IL-8 protein, Carcino Embryonic Antigen (CEA) protein, alpha 1 Fetoprotein (AFP), p53 protein, and spike protein (S-protein). In some embodiments, the target analyte can include an antibody selected from an anti-S-protein antibody, anti-troponin I antibody, anti-troponin T antibody, Anti-IgE antibody, anti-BNP antibody, Anti-BDNF antibody, anti-p53 antibody, anti-AFP antibody, anti-CEA antibody, anti-TRX antibody, anti-IL-8 antibody, and anti-IL-6 antibody.

As used herein, the term “small molecule” can refer to a natural or synthetic molecule having a molecular mass of less than about 900 Da. Examples of small molecules can include anhydrotetracycline (ATc), a small molecule toxin, a cannabinoid, to name a few.

As used herein, the term “macromolecule” can refer to a large molecule composed of thousands of covalently connected atoms such as carbohydrates, lipids, proteins, and nucleic acids to name a few examples. A macromolecule can be formed of repeating monomer units, forming a polymer. Macromolecules also include non-polymeric large molecules such as lipids (including phospholipids) and large macrocycles.

As used herein, the term “Inverted Molecular Pendulum” or “iMP” refers to the molecular systems or probes attached to the electrode of the electrochemical biosensor. In one embodiment, a plurality of iMPs can be attached to the electrode surface forming a monolayer of iMPs. Each iMP comprises at least a linker, a redox reporter and a receptor to receive a target analyte. Upon application of an electric field or current, the iMPs can be displaced towards the surface of the electrode. In one embodiment, the iMPs can present a relative rigidity, which can result in the inverted pendulum effect of the probes, which can “fall over” or “tilt” towards the electrode surface upon application of the electric field. The iMPs can be in two different states, an unbound state where no target analyte is bound to the receptor and a bound state where the target analyte is bound to the receptor.

As used herein, the term “linker” refers to a molecule that can be attached or anchored to an electrode surface and to which the receptor and the redox reporter of the iMP can be bound. The linker can be present a net positive or negative charge. The linker can assist in providing some rigidity to the iMP, which can allow the pendulum motion towards the biosensor surface upon an applied voltage. In some embodiments, the linker of the iMP is substantially rigid along the length of the linker. In some embodiments, the linker can have a length ranging from about 5 nm to about 20 nm. By linker length, one refers to the length of the linker itself and it therefore excludes the receptor length. Moreover, one should note that in order for the linker to be able to swing towards the biosensor surface, a certain flexibility should be present at the linker's end which is close to the electrode surface. Therefore, even if the linker is rigid almost all along its length, the extremity of the linker in the region close to the electrode surface can have some flexibility. This flexibility can be adjusted by selecting the suitable linker and/or by performing chemical modifications at the linker extremity close to the electrode surface. In certain embodiments, the linker can comprise a material such as double-stranded DNA (dsDNA), single-stranded DNA (ssDNA), charged polymers, uncharged polymers, or any combination thereof. In some embodiments, the linker can be negatively charged and can be a DNA/DNA duplex, a PNA/DNA duplex, a PNA/PNA duplex where one or both of the PNA are modified with negative charged amino acids such as with aspartic acids, a rigid anionic polyelectrolyte or a rigid negatively charged peptide. In some embodiments, the rigid anionic polyelectrolyte can be an anionic polyelectrolytic chain made rigid by its polymer architecture or by double layer forces of the solvent (e.g., poly(2-acrylamido-2-methylpropanesulfonic acid-co-acrylic acid). In some embodiments, the rigid negatively charged peptide can be a negatively charged peptide made rigid by its polymer architecture or by double layer forces of the solvent (e.g., double-stranded peptide with aspartic acid). In some embodiments, the linker can be positively charged and can be a PNA/PNA duplex with lysines, a rigid cationic polyelectrolyte or a rigid positively charged peptide. In some embodiments, the rigid cationic polyelectrolyte can be a cationic polyelectrolytic chain made rigid by its polymer architecture or by double layer forces of the solvent (e.g., Poly-y-benzyl-L-glutamate and poly[2-(methacryloyloxy)ethyl trimethylammonium chloride]. In some embodiments, the rigid positively charged peptide can be a positively charged peptide made rigid by its polymer architecture or by double layer forces of the solvent (e.g., double-stranded peptide with lysine). In one embodiment, the linker can comprise a dsDNA and the length of the linker can range from about 15mer to about 60mer. In another embodiment, the linker can include a ssDNA having a length ranging from about 10mer to about 100mer. As mentioned above, in some embodiments, the linker can be chemically modified to increase its flexibility near the electrode surface. Examples of chemical modifications can include the removal of nucelotides such as on the P2 strand of a dsDNA, to reveal single bases on the P1 strand. Hence, in one embodiment, when the linker includes a dsDNA, a first strand of the DNA is bound to the surface of the biosensor electrode and the second DNA strand can be modified by removing nucleotides from the 3′ end thereof to define a flexibility region at the extremity of the linker close to the electrode surface. The number of nucleotides that can be removed, can be adjusted such that the difference between the number of nucleotides in the first DNA strand and the number of nucleotides in the second DNA strand is from 1 to 15. In some embodiments, increasing the flexibility of the linker, and therefore iMP, by removing nucleotides from the 3′ end of the DNA strand not tethered to the surface can allow for increased signal output and detection resolution. Other possible chemical modifications can be contemplated in order to increase the linker flexibility near the electrode surface. For instance, it may be possible to introduce carbon atoms either at the thiol end of the P1 strand of the dsDNA or internally between bases near the thiol end. It can also be possible to use a poly-T chain, which is known to be more linear, but can also introduce more flexibility. Poly-T can be practical as it does not adsorb to gold as easily compared to a poly-A chain and is more flexible as a chain than poly-G or poly-C chains. In further embodiments, the length of the linker can be adjusted for increasing the signal output and detection resolution of the biosensor. For instance, when the linker comprises a DNA sequence, such as ssDNA and dsDNA, it was observed that, in a range of lengths, longer sequences can give decreased signal output and detection resolution.

As used herein, the term “receptor” refers to a molecular entity that is capable of binding an analyte, i.e., the target analyte. The receptor can also be referred to as “recognition agent”, “recognition element” or “capture agent”. The receptor is different than the target analyte and complementary to the target analyte. The receptor can be bound to at least a portion to the target analyte when the iMP comes into contact with the target analyte. In some embodiments, the receptor can comprise antibodies, nanobodies, antigens, aptamers, molecular imprints, protein receptors, DNA, microorganisms or protein/enzyme substrates. In one embodiment, the receptor can include a protein, an antibody or an aptamer. For instance, the receptor can be an antibody such as an anti-MRSA antibody, anti-MSSA antibody, anti-E. coli antibody, anti-Tuberculosis antibody, anti-pseudomonas aeruginosa antibody, anti-S-protein antibody, anti-troponin I antibody, anti-troponin T antibody, anti-IgE antibody, anti-BNP antibody, anti-BDNF antibody, anti-p53 antibody, anti-AFP antibody, anti-CEA antibody, anti-TRX antibody, anti-IL-8 antibody, or anti-IL-6 antibody. In some embodiments, the receptor can include a protein such as S-protein, Brain natriuretic peptide (BNP) protein, troponin I protein, troponin T protein, Natural Human IgE protein, Brain-derived neurotrophic factor (BDNF) protein, Thioredoxin (TRX), IL-6 protein, IL-8 protein, Carcino Embryonic Antigen (CEA) protein, alpha 1 Fetoprotein (AFP), or p53 protein. In some embodiments, the receptor can include a receptor binding domain (RBD) binding IgG. The receptor can include an aptamer sequence binding the RBD site of an S-protein. In other embodiments, the aptamer can include a BNP-specific aptamer. In other embodiments, the receptor can comprise a protein, an aptamer or an antibody specific to the SARS-CoV-2 spike protein. In other embodiments, the receptor can comprise SARS1 polyclonal anti 51 protein antibody, SARS1 51 protein, Receptor binding domain (RBD) binding IgG, SARS2 polyclonal anti S1 protein antibody, SARS2 Monoclonal anti S protein antibody, SARS2 Polyclonal anti S1 protein antibody, SARS2 S1 protein, or SARS2 S1 protein. In further embodiments, the receptor can be an aptamer targeting the Receptor-Binding Domain of the SARS-CoV-2 spike.

As used herein, the term “redox reporter” refers to a chemical entity that can bind to the linker of the iMP and can involve an electron transfer as the iMP approaches the electrode surface upon the effect of the pendulum falling over towards the electrode surface. In some embodiments, the redox reporter can touch the electrode surface and the electron transfer can be based on a redox reaction or electron tunneling current. In some embodiments, the redox reporter can observe a state change at a positive electric potential sufficiently close, but below the applied electric potential, such that the electron transfer rate is equivalent to the time-dependent rate at which the iMP falls over. In some embodiments, the redox reporter is bound to the linker at a distal end of the iMP, meaning in an upper portion of the linker opposite to the portion linked to the electrode surface. In some embodiments, the redox reporter can be covalently bound to the linker. The redox reporter can also be referred to as “redox transporter”. A non exhaustive list of redox reporters that can be present comprise ferrocene, [Co(GA)₂(phen)] (GA=glycolic acid, phen=1,10-phenathroline), metal nanoparticles (e.g., Au, Pt, Pd, Ag, Cu), pyrroloquinoline quinone (PQQ), benzoquine, Osmium(III) complexes such as Os(bpy)Cl₂³⁺, diphenylamine, methylene blue, Ruthenium(III) complexes such as Ru(NH₃)₆³⁺, neutral red, toluidine blue, or phenosafranine.

As used herein, the term “electrode” refers to any electrode or electrochemical system that is sufficient for detecting the change in electrochemical potential when an electron transfer from the redox reporter to the electrode occurs, namely upon binding of the target analyte to the receptor. The electrode can be a nanostructured electrode, a non-nano structured electrode, a micro-patterned electrode, or an array thereof. In some embodiments, the electrode can be a glassy carbon electrode, a carbon nanotube-modified electrode, an indium tin oxide (ITO) electrode, a platinum electrode, a gold electrode, a silver electrode, or a palladium electrode. The electrode can be fabricated on solid substrates including glass and silicon, or on flexible substrates including polyester (PET), polyimide (PI), polyethylene naphthalate (PEN), polyetherimide (PEI), optionally along with various fluoropolymers (FEP), copolymers, and paper. In some embodiments, the electrode can be a gold nanostructured microelectrode. In some embodiments, the electrode can be in the form of a wire. The wire can for example have a diameter between about 0.1 to about 1 millimeter. In other embodiments, the wire electrode can be embedded within a matrix of a non-conductive material such as a silicon resin (e.g., cured polydimethylsiloxane).

As used herein, the term “electrochemical signal” refers to the signal generated upon the electron transfer from the redox reporter of the biosensor to the electrode (i.e., change in current). In some embodiments, the electrochemical signal can represent the change in electron transfer decay rate between bound and unbound target analyte following application of a step voltage to the electrode. In some embodiments, the change in electron transfer decay can be performed by chronoamperometry.

As used herein, the term “sample” means any sample comprising or being tested for the presence of one or more target analyte. In some embodiments, the sample can be any biological fluid (e.g., from the body of a mammal or any other animal), including but not limited to blood, plasma, serum, saliva, urine, tears, sweat, to name a few examples.

### Biosensor

In one aspect, there is provided a biosensor that can be compatible with the analysis of various analytes, such as analytes present in biological fluids. For instance, the analytes can include proteins that are important physiological markers of disorders or diseases such as stress, allergy, cardiovascular health, inflammation or cancer, to name a few examples. The mechanism by which the biosensor can perform can be based on field-induced directional diffusion of complexes tethered on an electrode surface and the sensitivity of electron transfer reaction kinetics to molecular size, charge, and mass. This sensing mechanism can be compatible with making measurements in various biological fluids such as blood, saliva, faeces, urine, tears and sweat for instance. The biosensor can thus detect the target analyte and collect data “in situ”, meaning that the sensor can be deployed in the environment in which the analyte naturally exists. The collection of data can be made in living animals or in humans. The biosensor platform described herein can enable a broad range of applications in personalized health monitoring.

The biosensor can be designed as a monolayer of probes on an electrode surface, each probe being designed to behave as an Inverted Molecular Pendulum (iMP). In some embodiments, such biosensor can be reusable and is advantageously reagentless.

In some embodiments, the transient behaviour of the iMPs monolayer can be used as an indicator of whether an analyte has bound to the probe layer. Specificity can be achieved through the specific binding of target analytes to molecular receptors present on the iMPs. By assessing the dynamics of an iMP using first principles, one can understand the fundamental forces acting against the iMPs in an electrochemical system. As these forces would act on an iMP in solution (i.e., the biological fluid) redox chemistry can be used to take a quantitative measure of the iMP's transient motion. This phenomenon can require transient motion of the iMPs molecular monolayer to be simultaneous with electron transfer from the redox reporter molecule.

An iMP showing dynamic behaviour upon application of an electric field was therefore designed. In some embodiments, such an iMP's design can allow to sense a current upon application of the field at a known instant in time. The modelled iMP includes a linker anchored to an electrode, a receptor capable to bind to a target analyte positioned at the extremity of the linker which is not bound to the electrode and a redox reporter. In some embodiments, the linker can be a rigid linker anchored to the electrode surface and the linker can rotate or tilt within a non-linear electric field produced by applying an electric potential to the electrode surface.

In some embodiments, the biosensor can be characterized in that once the target analyte is bound to the receptor at the applied electric field/potential, an electrochemical signal can be produced which translates a difference between the electron transfer rate observed when no target analyte is bound to the receptor and the electron transfer rate where the target analyte is bound to the receptor. In some embodiments, the electron transfer (either in an unbound or bound state) can be observed as the redox reporter and thus the iMPs approach the electrode surface.

In other embodiments, the electron transfer rate can be dependent on the iMPs motion time rate. In other words, the electron transfer rate can be dependent on the time rate at which the iMPs are displaced towards the electrode surface. Hence, in some embodiments, the electron transfer rate in an unbound state can be dependent on a time rate at which the unbound iMPs are displaced. Similarly, the electron transfer rate in a bound state can be dependent on a time rate at which the bound iMPs are displaced. In some embodiments, the iMPs motion or displacement towards the electrode surface can be defined as a tilting movement of the iMPs as it pivots about its attachment to the electrode surface, which result in the inverted pendulum effect.

In some embodiments, upon application of an electric field, the redox reporter of the iMPs of the biosensor can touch the electrode surface and the resulting electron transfer can be based on a redox reaction or electron tunneling current.

The redox reporter can be bound to the linker close to the second end thereof, meaning that the redox reporter is generally conjugated to the linker in an upper portion thereof opposite the linker portion attached to the electrode surface. The redox can be covalently bound to the linker.

The linker of the iMPs present in the biosensor can be of different nature depending on the type of sample to be tested. In some embodiments, the linker can comprise a double-stranded DNA (dsDNA), single-stranded DNA (ssDNA), charged polymers, uncharged polymers, or any combination thereof. In some embodiments, the linker can be negatively charged and can comprise a DNA/DNA duplex, a PNA/DNA duplex, a PNA/PNA duplex where one or both of the PNA are modified with negative charged amino acids, a rigid anionic polyelectrolyte, a rigid negatively charged peptide, or any combination thereof. In other implementations, the linker can be positively charged and can comprise a PNA/PNA duplex with lysines, a rigid cationic polyelectrolyte, a rigid positively charged peptide, or any combination thereof.

In some implementations, the nature of the redox reporter can be dependent on the net charge of the linker. Table 1 below provides examples of redox reporter that can be used in the iMPs of the biosensor when the linker is negatively charged. Table 2 provides examples of redox reporter that can be used in the iMPs of the biosensor, when the linker is positively charged.

TABLE 1
examples of redox reporter that can be used in
combination with a negatively charged linker

| Linker | |
|---|---|
| charge | Negative |
| Redox | >0 mV |
| reporter state | |
| change | |
| Acceptable | Ferrocene |
| reporters | [Co(GA)₂(phen)] (GA = glycolic acid, |
| | phen = 1,10-phenathroline) |
| | Metal nanoparticles (eg., Au, Pt, Pd, Ag, Cu) |
| | pyrroloquinoline quinone (PQQ) |
| | Benzoquine |
| | Os(III) complex Os(bpy)Cl₂³⁺ |
| | Diphenylamine |

TABLE 2
examples of redox reporter that can be used in
combination with a positively charged linker

| Linker | |
|---|---|
| charge | Positive |
| Redox | <0 mV |
| reporter state | |
| change | |
| Acceptable | Methylene blue |
| reporters | Ru(III) complex (e.g., (Ru(NH₃)₆³⁺) |
| | Neutral red |
| | Toluidine blue |
| | Phenosafranine |

In some embodiments, the length of the linker can range from about 5 nm to about 20 nm. In a specific embodiment, where the linker comprises a dsDNA, the linker's length can range from about 15mer to about 60mer. In another embodiment, where the linker comprises a ssDNA, the linker's length can range from about 10mer to about 100mer.

The electrode used in the biosensor can be any type of electrode known in the art and compatible with the nature of the sample to be tested. The electrode can allow for detecting the change in electrochemical potential when an electron transfer from the redox reporter to the electrode occurs, namely upon binding of the target analyte to the receptor. In some embodiments, the electrode can be a nanostructured electrode, a non-nano structured electrode, a micro-patterned electrode, a wire, or an array thereof. In some embodiments, the electrode can be a gold nanostructured microelectrode or a gold wire electrode.

In some embodiments, the biosensor can be designed to detect at least two different target analytes. Therefore, it can be possible to attach iMPs of different nature to the electrode surface compatible with the detection of different target analyte. In other words, the iMPs monolayer on the electrode surface can comprise different iMPs. One can understand that such iMPs can differ either by their linker, their receptor, their redox reporter or any combination thereof. For instance, the biosensor could include two types of iMPs having the same linker and redox reporter, but with a different receptor for targeting two different analytes. Alternatively, the biosensor could include two types of iMPs having different linkers and different receptors, the combination linker/receptor of each iMP's type being selected to target specific analytes. Many combinations could be possible. It could also be possible to vary the length of the linker in addition to vary any one of the linker, receptor and/or redox reporter nature.

### Biosensor Applications

As mentioned above, the biosensor can be useful in various applications involving the detection of an analyte in a sample, more particularly a biological fluid. In some embodiments, the biosensor can be applicable in real-world disease monitoring where a single device would need to be persistently, reliably monitoring biofluids. More particularly, thanks to the iMPs capabilities, the biosensor can be compatible to be used as a wearable or implantable device for continuous, unsupervised monitoring of disease biomarkers. Hence, the biosensor can be used for in situ detection of a target analyte but could also be used for in vivo detection.

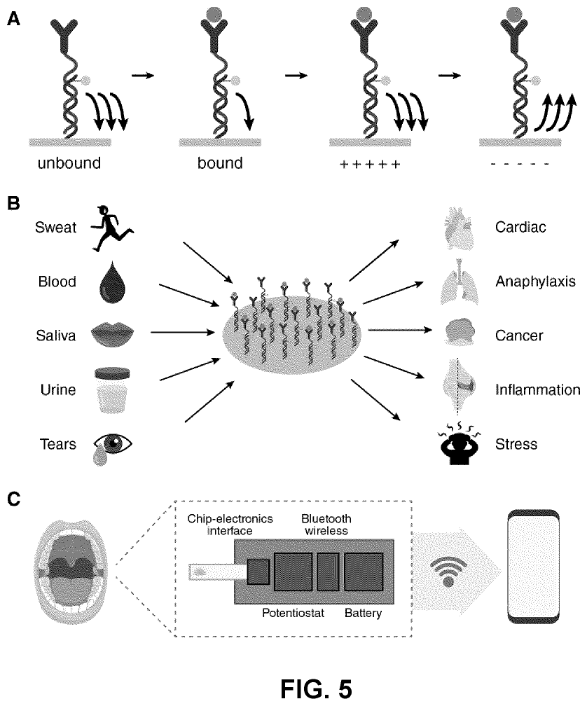

In some embodiments, the biosensor can be used as a wearable assay for real time electrochemical monitoring of disease (see e.g., FIGS. 5A-C, 18 and 19). Wearable technology has been on the rise lately with the development of new smart watches, health trackers, smart glasses, and other wearable technology. In some embodiments, the biosensor could be used in a broad range of applications in personalized health monitoring. In some implementations, the biosensor could be used for detecting a small molecule, a macromolecule, a prokaryotic or eukaryotic cell-derived component (e.g., nucleic acid material), a virus, a bacterium, an antibody, a protein, or a cellular extract in a sample. For instance, the biosensor could be used for performing the analysis of various physiological protein markers such as proteins involved in stress, allergy, infections, cardiovascular health, inflammation, and cancer. In addition, the biosensor is compatible with making measurements in different types of biological fluids and could this be used to detect analytes, such as proteins or viruses for instance, in blood, saliva, urine, faeces, tears and sweat. The system can be integrated into a disposable or a wearable device for real-time monitoring of disease. A disposable device comprising the innovative biosensor will be described below.

### Disposable Device

The present technology also concerns a disposable device for detecting a target analyte in a sample, such as a biological fluid, including the biosensor described above. In one embodiment, the disposable device can comprise a collector for collecting the sample, a sensing component comprising the biosensor according to the present technology and a connector for connecting the sensing component to an electrochemical measurement device. In some implementations, the electrochemical measurement device can be a handheld apparatus, which can enable rapidly assessing patients and individuals outside the hospital or medical clinic.

In some embodiments, the collector and the sensing component can be designed for allowing contact between the iMPs of the biosensor and the collected sample. In a further embodiment, the collector can be designed to at least partially encase the sensing component. The collector can be characterized in that it comprises a first portion and a second portion, where the first portion is able to contain the collected sample and the second portion can encase the sensing component.

In some embodiments, the first portion of the collector can comprise a nozzle and a sampling reservoir. The nozzle can either be patterned with microfluidic channels or filled with silica capillary tubes, in order to initiate passive capillary fluid flow once the nozzle comes in contact with the sample. In some implementations, the nozzle can comprise microfluidic channels which are treated with a hydrophilic coating.

As mentioned above, the sensing component of the disposable device includes the biosensor of the present technology and thus includes the iMPs connected to a first working electrode. In some embodiments, the sensing component can comprise further electrodes, such as two working electrodes (WE), a counter electrode (CE) and a reference electrode (RE). The two additional working electrodes can allow positive and negative controls to be determined within the same sensor from measuring at different working electrodes.

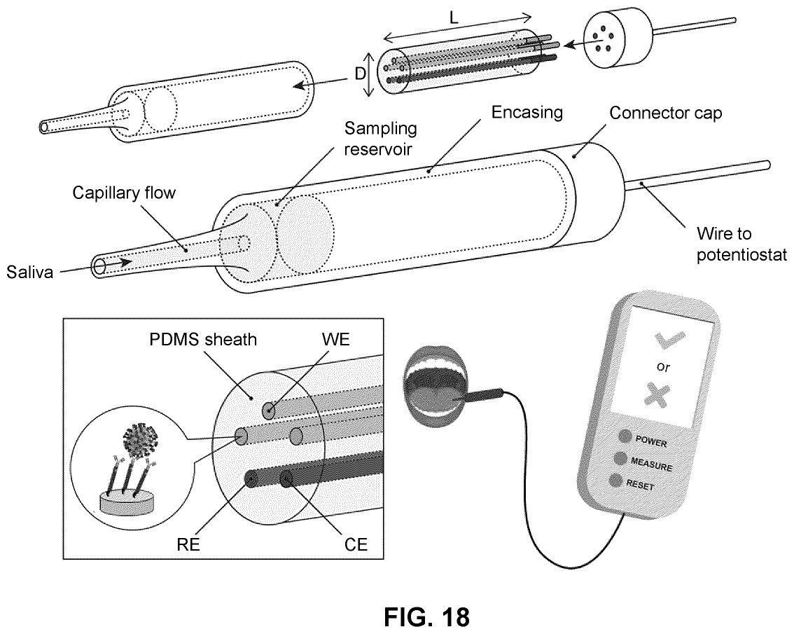

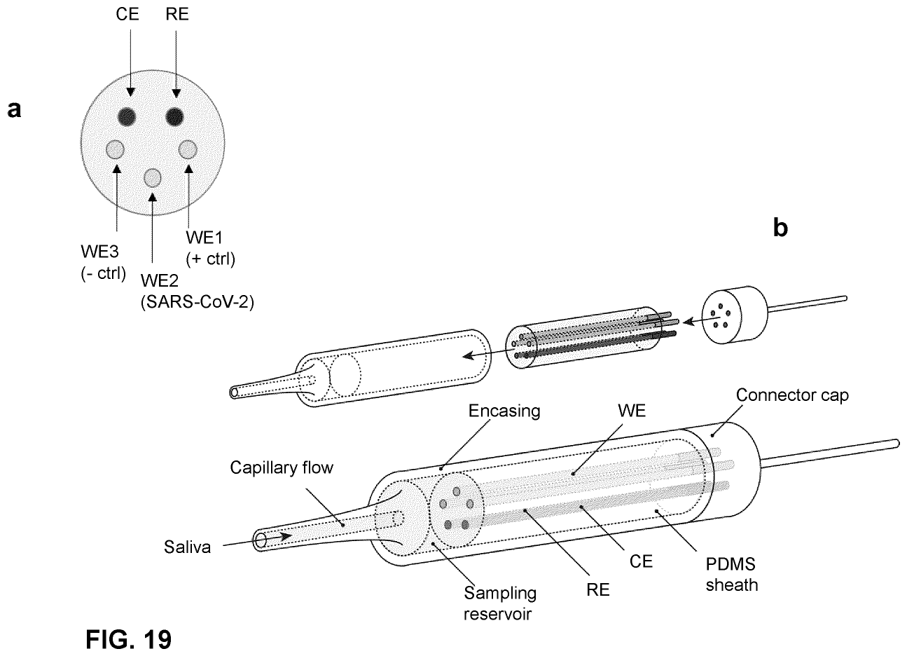

In some embodiments, the electrode of the biosensor and the two additional working electrodes can comprise gold. In another embodiment, the counter electrode can comprise platinum and the reference electrode can comprise silver. However, each electrode can either be solid gold, platinum or silver metal, or a plating/coating on an inexpensive metal. In some embodiments, each electrode can be in the form of a wire as shown for instance in FIGS. 18 and 19.

As also shown in FIGS. 18 and 19, the sensing component of the disposable device can have a cylindrical form with the electrode (e.g., electrode wires) positioned spaced apart within the sensing component along a length thereof. The cylinder forming the sensing component can have a length L and a diameter D. The length L and the diameter D of the cylinder can be adjusted. In some implementations, the length L can be from about 5 cm to about 10 cm (e.g., about 7 cm) and the diameter D can be from about 0.1 cm to about 1 cm, for instance about 0.5 cm. These values are provided only as examples and could be adapted depending on the whole design of the disposable device. In the case where the sensing component is in the form of a cylinder, the collector can thus have at least a portion thereof having a cylindrical form to properly encase the sensing component.

In other embodiments, the sensing component is designed such that the electrodes (e.g., electrode wires) are held in a matrix of a non-conductive material. Any non-conductive material commonly used in the field can be used to form the matrix holding the electrodes spaced apart. In some embodiments, the non-conductive material can be a silicon resin. For example, the non-conductive material can be a cured polyorganosiloxane, such as cured polydimethylsiloxane (PDMS). As shown in FIGS. 18 and 19, in the case where the electrodes are in the form of wires, the matrix can embed the wires which are parallelly positioned along the length of the cylinder, thereby forming a kind of shealth protecting the electrode wires.

The sensing component is also designed such that iMPs of the biosensor are in contact with the sample to be tested and which is collected in the collector element of the disposable device. In some implementations, the iMPs are bound to a first end of the biosensor electrode wire and the iMPs are exposed to the sample when the first end is in contact with the collected sample. In other implementations, each electrode wire (biosensor, WE, RE and/or CE) is in contact with the sample, at a first end thereof, when the sample has been collected in the collector. A second end of the wires can be in contact with the connector. In some implementations, the wires can extend from the extremity of the sensing component's matrix opposite to the extremity in contact with the sample, to allow the electronic connection with the connector and thus the electrochemical measurement device.

In some implementations, the connector of the disposable device can be in the form of a cap, which can connect to the extremity of the sensing component opposite the extremity in contact with the sample. In other implementations, the sensing component, which can be encased within the second portion of the collector can be secured within this second portion of the collector using a locking system. In some embodiments, the collector and the connector can be provided with complementary locking means to secure the sensing component within the device. The locking means can be any known system for fastening the second portion of the collector with the connector, for instance complementary screw threads, sealed joint, etc.

In some implementations, the extremity of the sensing component opposite the extremity in contact with the sample can also be closed off by an adapter, which can adapt the electrode wire connections to a plug and cable, which can connect to a handheld device (FIG. 18). The adapter can have holes to feed through the wire ends to larger metal terminals of the plug and can be fixed electrically using solder cup or crimp connections for instance.

In some implementations, the different elements of the disposable device can be fabricated using 3D printing.

The disposable device can provide an alternative to PCR-based testing and could accelerate the availability of high-quality diagnostic information. This can represent a tremendous advantage in monitoring a virus pandemic evolution for instance. Indeed, using the device can provide rapid, actionable diagnostic information on the pandemic status by facilitating serial and continuous monitoring of patients for the virus.

In some implementations, the biosensor and disposable device described herein can particularly be used to detect a virus selected from a coronavirus, adenovirus, filovirus, herpesevirus, poxvirus, parvovirus, reovirus, picomavirus, togavirus, orthomyxovirus, rhabdovirus, retrovirus, hepadnavirus, lentivirus, norovirus. In some embodiments, the biosensor and/or the device comprising the biosensor can be used to detect a coronavirus such as a SARS-CoV or a MERS-CoV virus, such as the SARS-CoV-2 virus. In another implementation, the biosensor to detect the SARS-CoV-2 virus can include a double-stranded DNA (dsDNA) linker and a protein, an aptamer or an antibody specific to the SARS-CoV-2 spike protein as receptor. In some implementations, the redox reporter can include ferrocene. In some implementations, the biosensor to detect the SARS-CoV-2 virus can include SARS1 polyclonal anti S1 protein antibody, SARS1 S1 protein, Receptor binding domain (RBD) binding IgG, SARS2 polyclonal anti S1 protein antibody, SARS2 Monoclonal anti s protein antibody, SARS2 Polyclonal anti S1 protein antibody, SARS2 S1 protein, or SARS2 S1 protein as the receptor. In some embodiments, the receptor is an aptamer targeting the Receptor-Binding Domain of the SARS-CoV-2 spike.

In a further implementation, the biosensor and disposable device described herein can be used to detect small molecules, such as cannabinoids (e.g., THC, CBD, etc.). The disposable device as described herein, can be used by the police authorities to test an individual (e.g., a driver) to assess if that person is under the influence of cannabis. Hence, the biosensor and/or the device comprising the biosensor can be used to detect cannabinoids, e.g., THC, in a biological fluid. In one implementation, the biosensor can include an aptamer or an antibody as the receptor for detecting cannabinoids.

As can be noted, the biosensor and disposable device described herein can be advantageously used for the detection of different types of analytes in a sample. Although examples of analytes that can be detected using the present biosensor have been provided herein, the technology is not limited to such analytes and the biosensor can be designed by varying the linker, redox reporter and receptor to allow detection of a large variety of analytes.

### EXPERIMENTAL TESTINGS

### Example 1

### Sensor Fabrication and Protein Detection

### Materials and Methods

Materials. Probe sequences used were: 5′-SH-MC6-TAC CAG CTA TTG TAT CTA ATA AGA-NH₂-3′ and 5′-NH₂-C6-TCT TAT TAG ATA CAA TAG CTG GTA. All the DNA sequences were obtained from Integrated DNA Technologies (IDT). Ferrocene NHS ester was obtained from Five Photon Biochemicals. All the antibodies and proteins were obtained from AbCam except Rantes antibody and protein that were obtained from R&D systems. Cardiac Troponin I protein (CTI), anti-CTI antibody, Recombinant Human Cardiac Troponin T protein (CTT), Anti-CTT antibody, Natural Human IgE protein, Anti-IgE antibody, Recombinant Human Brain natriuretic peptide (BNP) protein, Anti-BNP antibody, Recombinant Human/Murine/Rat Brain-derived neurotrophic factor (BDNF) protein, Anti-BDNF antibody, Recombinant Human p53 protein, Anti-p53 antibody, Recombinant Human alpha 1 Fetoprotein (AFP), Anti-AFP antibody, Recombinant Human Carcino Embryonic Antigen (CEA) protein, Anti-CEA antibody, Recombinant human Thioredoxin (TRX), Anti-TRX antibody, Recombinant human IL-8 protein, Anti-IL-8 antibody, Recombinant human IL-6 protein, Anti-IL-6 antibody were obtained from AbCam. Sterile PBS and UltraPure DNase/RNase-Free Distilled Water were obtained from Wisent Bioproducts. Saliva, tear, urine, and sweat were obtained from Lee Biosolutions. Human blood and urine were collected from healthy volunteers. Doxorubicin hydrochloride, Tris(2-carboxyethyl)phosphine hydrochloride (TCEP), and 6-Mercapto-1-hexanol (MCH), HAuCl₄ solution was obtained from Sigma-Aldrich.

Ferrocene conjugation. Ferrocene was conjugated to the amine-terminated DNA sequences by following the protocol supplied by the company (Five Photon Biochemicals). Briefly, ferrocene-NHS Ester (9.85 mg, 30.11 micromoles) was dissolved in 1.0 milliliter of methyl sulfoxide and 3 micromoles amino-oligonucleotide was dissolved in 800 microliters of 0.2 M sodium carbonate buffer (pH 8.5). The ester solution (400 microliters) was added to the amino-oligonucleotide solution. The mixture was incubated for 4 hours at room temperature or 16 hours at 4° C., after which it was purified by using chromatography on a Sephadex™ G-25 column using de-ionized water/carbonate buffer (50/50) as eluent. The fraction with yellow color was dialyzed against water to remove excess salts and unreacted reagents.

Antibody conjugation. Antibody was conjugated to the amine-terminated DNA sequences by using antibody-oligonucleotide conjugation kits obtained from solulink (Version 12.12.2012) and AbCam (ab218260, version 2).

Fabrication of the chips and formation of sensors. Fabrication of the chips and growth of the nanostructured sensors were performed as described previously in B. Lam, R. D. Holmes, J. Das, M. Poudineh, A. Sage, E. H. Sargent, S. O. Kelley, Lab Chip 2013, 13, 2569-75, with little modifications. Briefly, glass chips were fabricated in-house utilizing substrates obtained from Evaporate Metal Films, Inc. (Valencia, Ithaca, N.Y.) that were pre-coated with 5 nm Cr-100 nm Au and S1811 positive photoresist (MicroChem, Newton, Mass.) was spin-coated onto the substrates in-house (4500 rpm, 90 s). Sensing electrodes were patterned using standard photolithography and etched using Au and Cr wet etchants followed by removal of the positive photoresist etchant mask. A negative photoresist SU-8 2002 (3000 rpm, 60 s) was spin-coated to create 20-μm apertures. Chips were diced in-house using a standard glass cutter and were rinsed with acetone, isopropyl alcohol, and deionized (DI) water. After cleaning, the chips were dried with a flow of nitrogen before use. Sensors were electroplated in apertures in a solution of 50 mM HAuCl₄ and 0.5 M HCl using direct current (DC) potential amperometry at a constant potential of 0 mV for 100 s. A fine nanostructured Au coating was formed on the Au structure during a second electrodeposition step in the same solution of Au at constant potential of −450 mV for 10 s (J. Das, S. O. Kelley, Anal. Chem. 2013, 85, 7333-7338).

Sensor functionalization. A 1 μM thiolated probe solution in PBS was mixed with TCEP for disulphide reduction and incubated for an hour in a dark chamber. This thiolated probe contained a thiol (SH) group at 5′ end and a ferrocene redox reporter at the other (3′) end. Probe solution was heated to 55° C. for 5 min and chilled. After that, 1 μM of the antibody-conjugated complementary probe was mixed to this thiolated probe mixture and incubated for 10 min for hybridization. A 9 μM of 6-Mercapto hexanol (MCH) solution was mixed with the probe mixtures. After that, the probe solution was dropped onto the chips and incubated for overnight in a dark humidity chamber at room temperature for immobilization of probe. The chip was then washed for 5 min with PBS at room temperature three times. Before testing, sensors were immerged into ten-times diluted PBS solution. After initial electrochemical scanning the chips were then treated with different targets at room temperature for 50 minutes. After target incubation, chronoamperometric experiments were performed in the same solution without washing the sensor.

Animal study. All animal experiments were conducted according to the protocol approved by University of Toronto Animal Care Committee, which follows the guidelines and standards of Canadian Council on Animal Care (CCAC).

6 to 8-week-old C57BL/6J male mice (Jackson Laboratory) were used in all studies. To induce acute cardiotoxicity, mice were injected intraperitoneally with Doxorubicine for 4 days and at day 5 saliva samples were collected or in-situ measurement was carried out in the control and treated mice. In some experiments, we administered cardiac troponin I (51 μg/mouse) via tail vein injection and signal was measured continuously in-situ. Saliva samples in different time points were also collected for in-vitro analysis. A control sensor was used for reference, in which DNA-probe was modified with BSA instead of an antibody.

Electrochemical analysis. Electrochemical experiments were carried out using a Bioanalytical Systems Epsilon Basi potentiostat (or a miniature electrochemical electronic) with three-electrode system featuring the sensor as working electrode, an Ag/AgCl (or on chip gold) as reference electrode and a platinum wire (or on-board gold) as a counter electrode. Electrochemical signal was recorded by using chronoamperometry using a potential window from 0 to +500 mV (vs. Ag/AgCl reference electrode) or −200 to +300 mV (vs. an on-board pseudo gold reference electrode) for 50 ms.

### Results

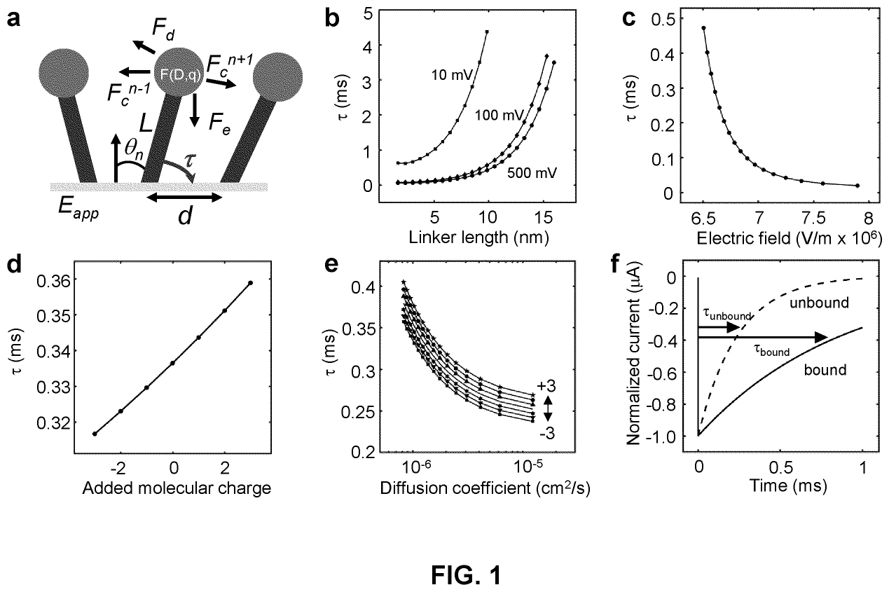

The modelled iMP was a rigid dsDNA linker, which has a net negative charge, anchored to the electrode and allowed to rotate within a non-linear electric field produced by applying a positive potential to the electrode surface (FIG. 1a). As the iMP drifts through the field, hydrodynamic drag and electrostatic interactions between neighbouring iMPs, as well as the electric force pulling the iMP downwards, can modulate the time required for the sensor to interact with the electrode surface. This characteristic time can be observed by having a redox reporter molecule conjugated to the distal end of the iMP, causing an electron transfer as the iMP approaches the electrode. For this to happen, a redox reporter can be selected, which observes a state change at a positive potential sufficiently close, but below the applied potential, such that the electron transfer rate can be equivalent the time-dependent rate at which the iMPs fall. Equations for the iMP's motion can be developed from first principles, and the characteristic time, τ, equivalent to the electron transfer rate k=1/τ, can be expressed by taking a first order approximation as:

$$τ = \frac{{ϵ}_{r} L {k}_{B} T}{D \vert qE \vert}$$

This equation can allow the experimentalist to make an informed assessment for iMP designs based on the analyte detection of interest. To observe analytes at the protein level, it was found that the temporal response could lay in the experimentally accessible microsecond-to-millisecond regime when the length of the linker was in the range 3-10 nm (FIG. 1b). Longer probes were slower to respond and exhibited weaker responses since the field was decayed when the charged entity was above about 10 nm from the electrode. With values for linker length and applied potential in place, one can test how modulating the electric field by increasing solution ionic strength (FIG. 1c) and molecular charge of an analyte (FIG. 1d) can affect the τ values, determining that solution and analyte charge differences can modulate observed response. It was then observed that small changes in both charge and analyte diffusional coefficient, a function of analyte size, in a relevant range for protein detection show readily distinguishable changes in using this approach (FIG. 1e). Since τ is the first-order response of a time-invariant system, it was equated to the exponential decay of the field-induced electrochemical response of a monolayer of iMPs in the form Aₒe^{-t/τ}. By this, the change in electron transfer decay rate between bound and unbound sensors by applying a step voltage to the electrode can be detected (FIG. 1f). The amplitude of the signal at τ, corresponding to the current measurement at that time, can then be determined, making it easy to differentiate signals of bound and unbound sensors.

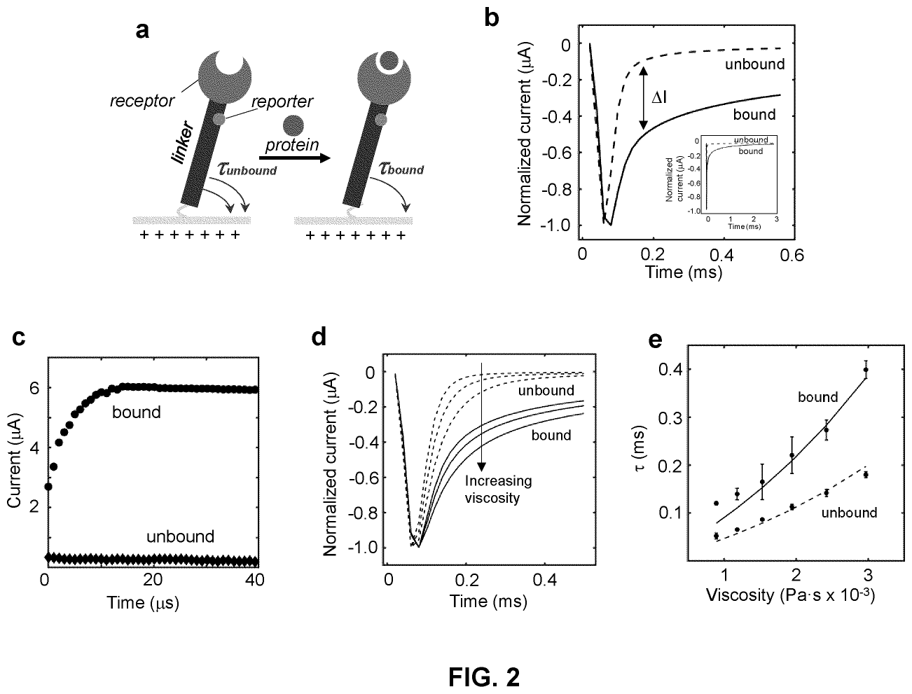

Next, a protein-binding iMP was constructed on a gold nanostructured microelectrode (NME) using a DNA linker with an antibody conjugated at its distal end specific to Cardiac Troponin I (CTI), an important cardiac disease biomarker, with a ferrocene reporter (FIG. 2a). Chronoamperometry, a potential-stepping method, was used to observe changes in current (i.e., electron transfer decay) in the presence and absence of CTI (FIG. 2b), showing the changes as predicted by the iMP model. The current modulation was also observed as a function of time to observe the binding kinetics of CTI to the biosensor in solution, showing observable detection after about 15 minutes (FIG. 2c). Next, iMP dynamics in differing fluid matrices were explored by linearly increasing the viscosity of the buffer with added glycerol. iMP dynamics were observed in its unbound state and bound state using a secondary antibody to CTI to see a large effect (FIG. 2d). It was determined that the experimental and theoretical were in agreement, with higher τ for the bound state across increasing viscosity (FIG. 2e). These results provide insights into how the iMPs can be calibrated when deployed in different biofluids.

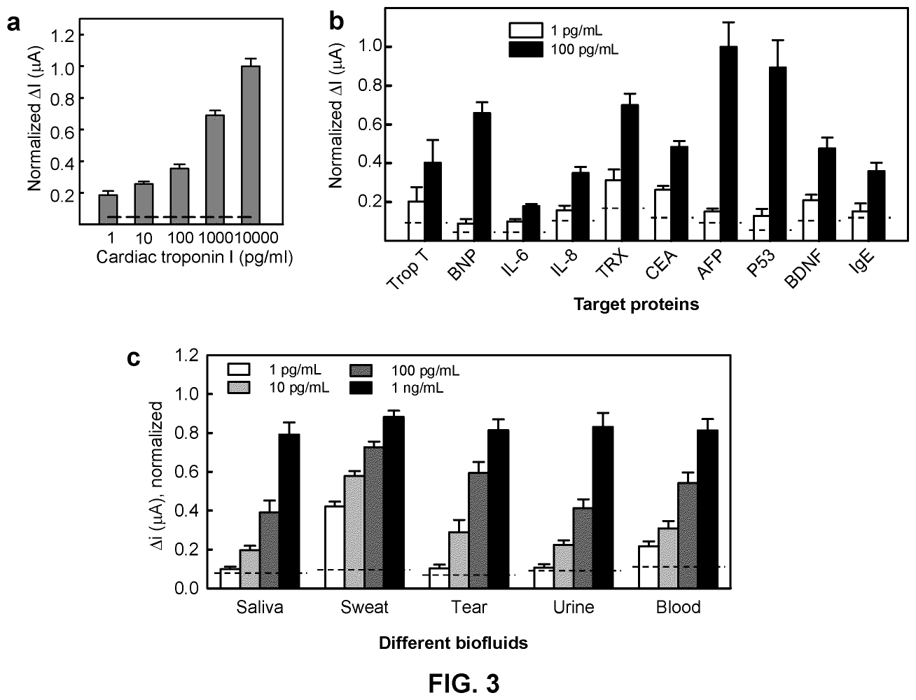

Continuing with the CTI case, detection down to 1 picogram/milliliter (˜40 femtomolar) concentrations of CTI was possible (FIG. 3a). The iMPs were also used to detect numerous other analytes specific to cardiac (CTT and BNP), inflammation (IL-6 and IL-8), stress (TRX), depression (BDNF), anaphylaxis (IgE) and cancer (CEA, AFP and P53) detection (FIG. 3b), demonstrating the universality of the present biosensing approach. Furthermore, again using CTI as a test case, detection of a range of concentrations of CTI in multiple biofluids was also rendered possible (FIG. 3c), demonstrating the iMPs versatility to many realistic modes of in situ monitoring for disease.

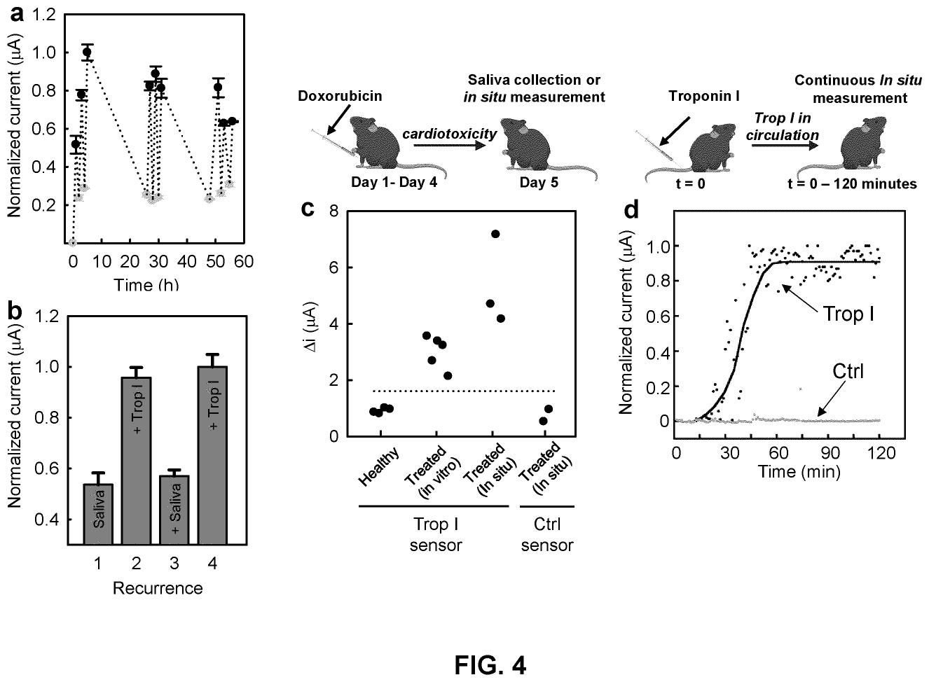

In order to demonstrate that the present technology could be used long-term, it was first demonstrated that the sensor can bind and unbind analytes over time depending on its concentration in the system (FIG. 4a). This means that the iMP system can detect both increasing and decreasing levels of analyte over time. The iMP system can also be reliability used after a long period of time (FIG. 4b). The sensors were kept in saliva for 3 weeks before being tested to detect CTI.

Next, two studies of non-invasive in situ measurements were performed to demonstrate disease monitoring in murine models. Cardiac dysfunction in mice was first induced through injections of doxorubicin and saliva samples were measured after 5 days. The iMP sensors were able to detect the CTI produced by the mouse, visualized by the change in current compared to the baseline healthy mouse for both in vitro and in situ models. Continuous monitoring of CTI in mice through injections of CTI and automating chronoamperometric measurements every 30 seconds for 2 hours was also performed. An increase of CTI in the mouse saliva over time was detected, demonstrating the iMPs capabilities as a wearable or implantable device for continuous, unsupervised monitoring of disease biomarkers.

### Example 2

### Viral Proteins and Particles Detection in Saliva

### Materials

DNA probe sequences used were: 5′-SH-MC6-TAC CAG CTA TTG TAT CTA ATA AGA-NH2-3′ (P1) and 5′-NH2-C6-TCT TAT TAG ATA CAA TAG CTG GTA (P2). Aptamer sequence binding the receptor binding domain (RBD) site on S1 protein subunit (sequence from Song, Y. et al. Discovery of Aptamers Targeting Receptor-Binding Domain of the SARS-CoV-2 Spike Glycoprotein. Anal. Chem. 2020, 92, 14, 9895-9900. [Cov2-RBD-4C]). The aptamer-P2 sequence used was ATC CAG AGT GAC GCA GCA TTT CAT CGG GTC CAA AAG GGG CTG CTC GGG ATT GCG GAT ATG GAC ACG TTT TTC TTA TTA GAT ACA ATA GCT GGT A. All the DNA sequences and aptamer were obtained from Integrated DNA Technologies (IDT). Ferrocene NHS ester was obtained from Five Photon Biochemicals.

Proteins and antibodies used as capture agents (receptors) for detection of viral proteins and viral particles:
1. SARS1 polyclonal anti-S1 protein antibody from AbCam (Cat: ab252690)
2. SARS1 S1 protein from AbCam (Cat: ab49046)
3. Receptor binding domain (RBD) binding IgG (generated at Rini laboratory-UofT, binds both SARS1 and SARS2 S1 proteins)
4. SARS2 polyclonal anti-S1 protein antibody from AbCam (Cat: ab272504)
5. SARS2 Monoclonal anti-S protein antibody from Sinobiological (Cat: 40150-R007)
6. ARS2 Polyclonal anti-S1 protein antibody from Genetex (Cat: GTX135356)
7. SARS2 S1 protein from AbCam (Cat: ab272105)
8. SARS2 S1 protein from Genetex (Cat: GTX01548-pro)

### Pseudotyped Viral Particle:

1: Lentivirus expressing S protein on its membrane.
2: Lentivirus similar to previous sample without expressing S protein—used as negative control.

### Methods

Ferrocene conjugation via NHS ester chemistry. Ferrocene was conjugated to the amine-terminated DNA sequences by following the protocol supplied by the company (Five Photon Biochemicals). Briefly, ferrocene-NHS Ester (9.85 mg, 30.11 micromoles) was dissolved in 1.0 milliliter of methyl sulfoxide and 3 micromoles amino-oligonucleotide was dissolved in 800 microliters of 0.2 M sodium carbonate buffer (pH 8.5). The ester solution (400 microliters) was added to the amino-oligonucleotide solution. The mixture was incubated for 4 hours at room temperature or 16 hours at 4oC, after which it was purified by using chromatography on a Sephadex™ G-25 column using de-ionized water/carbonate buffer (50/50) as eluent. The fraction with yellow color was dialyzed against water to remove excess salts and unreacted reagents.

Ferrocene conjugation via copper-catalyzed click chemistry. DNA sequences (Integrated DNA Technologies) modified with a thiol group on the 5′ end and an azide group on the 3′ end was used for the conjugation of ferrocene to the 3′ end. A fresh stock of 0.1 M copper bromide (CuBr) solution in DMSO was prepared before each conjugation. A 0.1 M tris((1-benzyl-4-triazolyl)methyl)amine (TBTA) solution in DMSO was prepared and stored at −20° C., and was thawed before use. A ratio of 1:2 (v/v) of CuBr to TBTA was combined to make the click solution. A 200 pM solution of DNA in deionized water was prepared. Separately, a 50 mM solution of ethynyl ferrocene (Chemscene in DMSO) was prepared. Ethynyl ferrocene was added to the DNA solution to a final concentration of 2 mM. Finally, 3 μL of the click solution was added to a 5 μL of the 200 μM DNA solution. The final combined solution was gently shaken to react at room temperature for 3 hours. After this time, the reaction was diluted with 100 μL of 0.3 M sodium acetate. The DNA was subsequently precipitated with cold ethanol and centrifuged at 15,000 rpm for 15 min to form a pellet. The supernatant was removed and the pellet was washed and centrifuged 3 times with cold ethanol. The pellet was finally resuspended in water before further purification by HPLC.

Antibody and protein conjugation. Antibody and protein were conjugated to the amine-terminated DNA sequences (P2) by using antibody (or protein)-oligonucleotide conjugation kits obtained from solulink (Version 12.12.2012) and Abcam (ab218260, version 2).

### Fabrication of the Chips and Formation of Sensors

Fabrication of the chips and growth of the nanostructured sensors were performed as described previously in B. Lam, R. D. Holmes, J. Das, M. Poudineh, A. Sage, E. H. Sargent, S. O. Kelley, Lab Chip 2013, 13, 2569-75, with little modifications. Briefly, glass chips were fabricated in-house utilizing substrates obtained from Evaporate Metal Films, Inc. (Valencia, Ithaca, N.Y.) that were pre-coated with 5 nm Cr-100 nm Au and S1811 positive photoresist (MicroChem, Newton, Mass.) was spin-coated onto the substrates in-house (4500 rpm, 90 s). Sensing electrodes were patterned using standard photolithography and etched using Au and Cr wet etchants followed by removal of the positive photoresist etchant mask. A negative photoresist SU-8 2002 (3000 rpm, 60 s) was spin-coated to create 20-μm apertures. Chips were diced in-house using a standard glass cutter and were rinsed with acetone, isopropyl alcohol, and deionized (DI) water. After cleaning, the chips were dried with a flow of nitrogen before use. Sensors were electroplated in apertures in a solution of 50 mM HAuCl₄ and 0.5 M HCl using direct current (DC) potential amperometry at a constant potential of 0 mV for 100 s. A fine nanostructured Au coating was formed on the Au structure during a second electrodeposition step in the same solution of Au at constant potential of −450 mV for 10 s (J. Das, S. O. Kelley, Anal. Chem. 2013, 85, 7333-7338).

Sensor functionalization. A 1 μM thiolated probe solution (P1 probe-conjugated to ferrocene) in PBS was mixed with TCEP for disulphide reduction and incubated for an hour in a dark chamber. This thiolated probe contained a thiol (SH) group at 5′ end and a ferrocene redox reporter at the other (3′) end. Probe solution was heated to 55° C. for 5 min and chilled. After that, 1 μM of the antibody (or protein)-conjugated complementary probe or aptamer-P2 was mixed to this thiolated probe mixture and incubated for 10 min for hybridization. A 9 μM of 6-Mercapto-1-hexanol (MCH) solution was mixed with the probe mixtures. After that, the probe solution was dropped onto the chips and incubated for overnight in a dark humidity chamber at room temperature for immobilization of probe. The chip was then washed for 5 min with PBS at room temperature three times. Before testing, sensors were immerged into ten-times diluted PBS solution. After initial electrochemical scanning the chips were then treated with different targets at room temperature for different time. After target incubation, chronoamperometric experiments were performed in the same solution without washing the sensor.

Viral particle preparations. Viral particles are stored at −80° C. and defrosted before use. Viral particles were stored in culture media. They were diluted with 0.1×PBS, 1×PBS, or human saliva). The initial concentration of the samples was calculated with p24 ELISA kits (HIV1 concentration) to be 10¹² particles/mL. Eleven (11) dilutions of the virus samples (10× dilution factor) were made to have access to various concentrations of virus in the proper buffer.

Electrochemical analysis. Electrochemical experiments were carried out using a Bioanalytical Systems Epsilon Basi potentiostat with three-electrode system featuring the sensor as working electrode, an Ag/AgCl as reference electrode and a platinum wire as a counter electrode. Electrochemical signal was recorded by using chronoamperometry using a potential window from 0 to +500 mV (vs. Ag/AgCl reference electrode) for 50 ms.

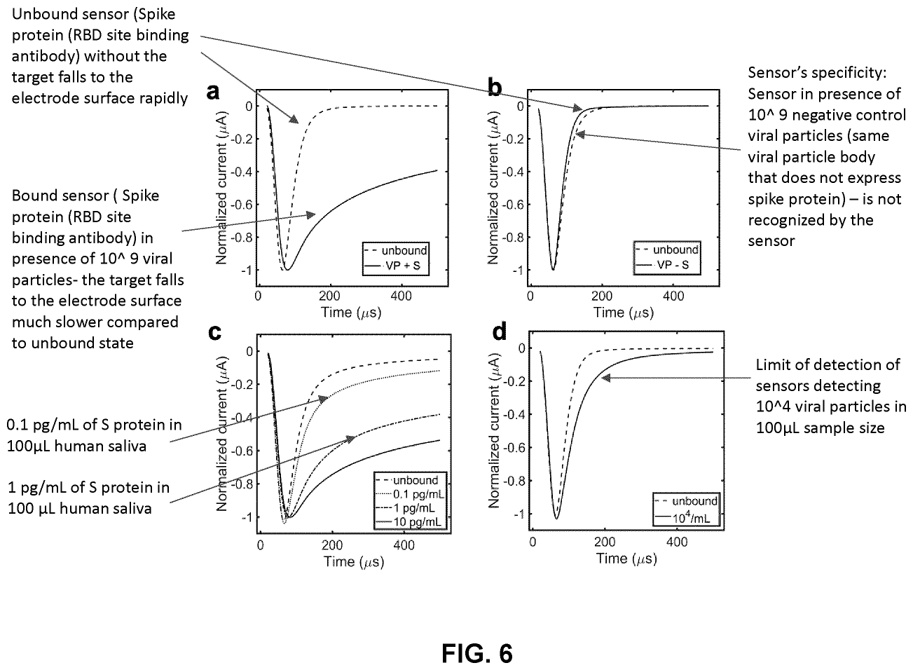

### Direct Viral Particle Detection (FIG. 6)

Sensor: RBD binding IgG from Rini lab, targets: viral particles from Cochrane lab, S protein from AbCam.

Direct detection of pseudotyped SARS viral particle expressing spike protein on their surface membrane in human saliva (FIG. 6a). After post immobilization washes, the sensor's chip was electrochemically tested under test buffer and human saliva. The current decay vs. time (chronoamperometry) was measured as the reference and is shown in the figure with dashed line (unbound sensor, i.e., sensor without target viral particle shows faster decay rate). Then, the same chips were incubated with 100 μL of 10⁹ particle/mL of the viral particles for 50 minutes. After the incubation period, the electrochemical signal was measured with the sensors with the same conditions. (shown in the figure with solid line). The lower rate shows the slower travel speed of the molecular pendulum to the surface of electrode due to its attachment to the viral particle. In a negative control (FIG. 6b), similar experiment as of FIG. 6a was performed with pseudotyped viral particles that do not express S proteins on their membrane. The current decay rate did not change compared to the unbound sensor, which demonstrates that the specific detection of viral particle through binding to the S protein. The Limit of detection of the sensor detecting SARS CoV S protein diluted in human saliva was determined (FIG. 6c). The s protein was diluted in human saliva at concentrations of 0.1 pg/mL, 1 pg/mL, 10 pg/mL. After reading the unbound state of the sensor electrochemically, 100 μL of 0.1 pg/mL sample was loaded on the chip and incubated for 50 minutes. After 50 minutes of incubation, the same chronoamperometry was performed and signal was readout. The chip was then washed with PBS (3 times wash). Next, 100 μL of the 1 pg/mL sample was loaded to the same chip. After 50 minutes incubation the read and wash steps were repeated and then the 10 pg/mL sample was loaded to the chip. Followed by incubation time, and final readout. Each read is a line in the figure, showing the decreasing decay rates after addition of high concentrations of protein. The sensor detects SARS CoV-2 pseudotyped viral particles diluted to concentrations of 10⁴ particles/mL in 100 μL samples (FIG. 6d). The viral particles were diluted to 10⁴ particle/mL in 0.1×PBS and loaded to the chip after the first readout in buffer (0.1×PBS). The dashed line represents signal without vital particles (unbound state), while the solid line represents the signal in presence of viral particles (bound state).

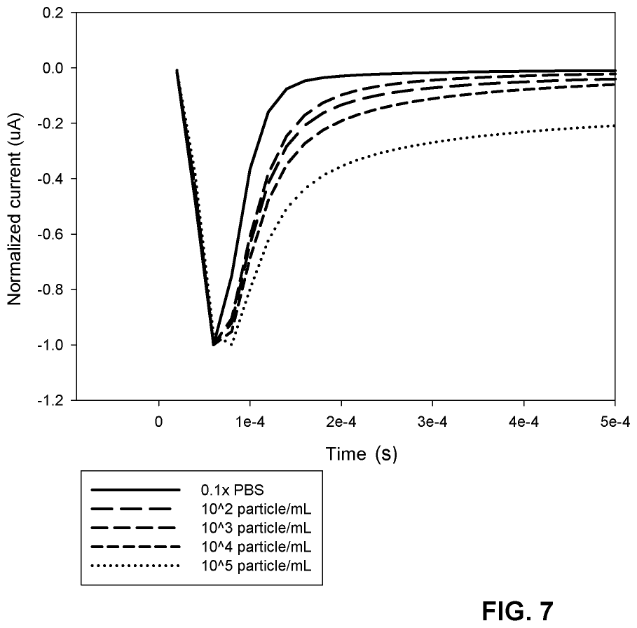

### Measuring the Limit of Detection of the Sensor Detecting the Viral Particles (FIG. 7)

The sensitivity of the system was evaluated for detection of viral particles (using RBD binding IgG from Rini lab as capture probe). Serial dilution of the viral particle sample was made. The chips were first measured in 0.1×PBS as unbound state (solid line in figure). Then the limit of detection of the system were measured following the same incubation, readout, wash steps as in FIG. 6b. The starting concentration for the viral particles was 10² particle/mL with 100 μL sample volume. Going to higher concentration of sample, the sensor successfully detected 100 μL of 10⁴ particles/mL viral particles.

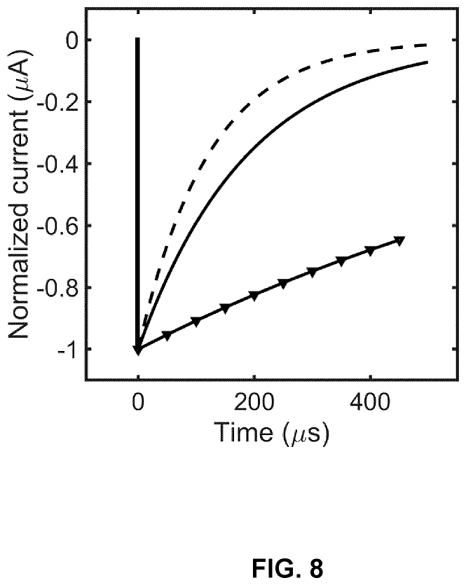

Modeling the Viral Particle Detection within the System (FIG. 8)

Comparison of decay curves of the unbound, isolated S-protein, and SAR-CoV-2 detection, in the order of increasingly shallower slope (FIG. 8). This indicates that the larger the bound analyte is, the shallower the current decay curve will be, as larger bound analytes take longer to fall due to competing hydrodynamic drag against the electrostatic pull between the DNA linker and electrode. In this case, the SARS-CoV-2 virus is much larger in size (˜100 nm) compared to isolated S-protein (<10 nm).

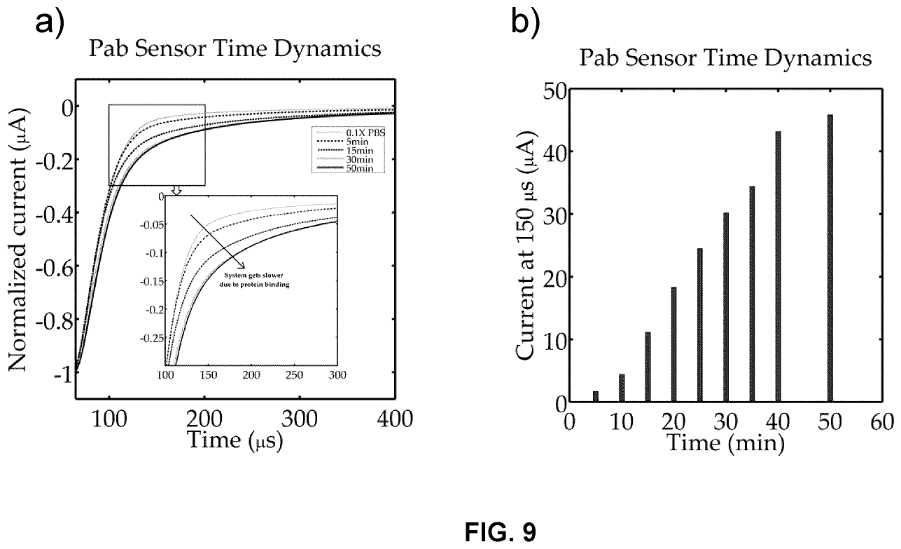

### Detection of the SARS-CoV-2 S Protein (FIG. 9)

For the real-time detection of S protein (kinetics of binding event), the polyclonal antibody (from AbCam) was used as the capture agent (receptor) and detection of S protein (from AbCam) was measured (FIG. 9). After the readout for unbound state of the sensor (dotted line on figure), 50 μL of the CoV-2 S protein at 1 ng/mL concentration was added to the chip (FIG. 9a). In order to assess the kinetics of protein detection, chronoamperometry (CA) was measured every 5 minutes and the decay rate of the current was measured. A bar graph representation of the real time measurement of S protein with time indicates signal generation in as little as 5 minutes (FIG. 9b). The absolute amount of current was selected at 150 μs after start of chronoamperometry. At this time-point, all the plots were compared to each other. The signal rise starts after 5 minutes of incubation demonstrating that signal can be detectable is a short incubation time.

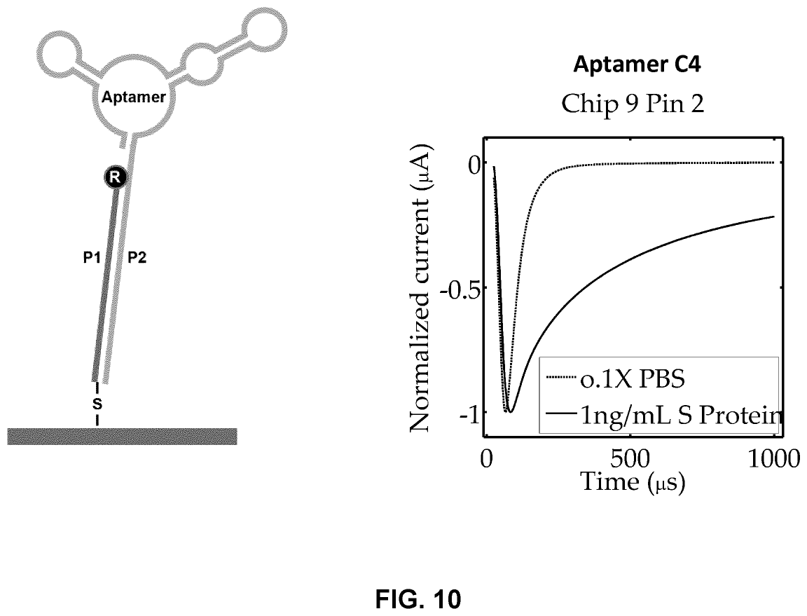

Detection of SARS-CoV-2 S Protein using an Aptamer Sensor (FIG. 10)

The sensor was reconfigured to use a DNA based aptamer for binding instead of an antibody. The capture agent (receptor) was changed to an RBD binding DNA probe (aptamer) and the efficiency of the system was evaluated. The aptamer-based probe sequence was designed in a way that 3′ end of the probe contains the P2 sequence which can hybridize to P1. Then, P1/aptamer-P2 duplex was immobilized on the NME gold surface. After the readout for unbound state in 0.1×PBS (solid line on graph), 0.1×PBS containing 1 ng/mL of the S protein was loaded on chips. After 50 minutes incubation, the secondary readout was measured (solid bold line), which shows significant decrease in decay rate of the current. This shows that depending on the binding affinity (Kd), the system can be made of various building blocks.

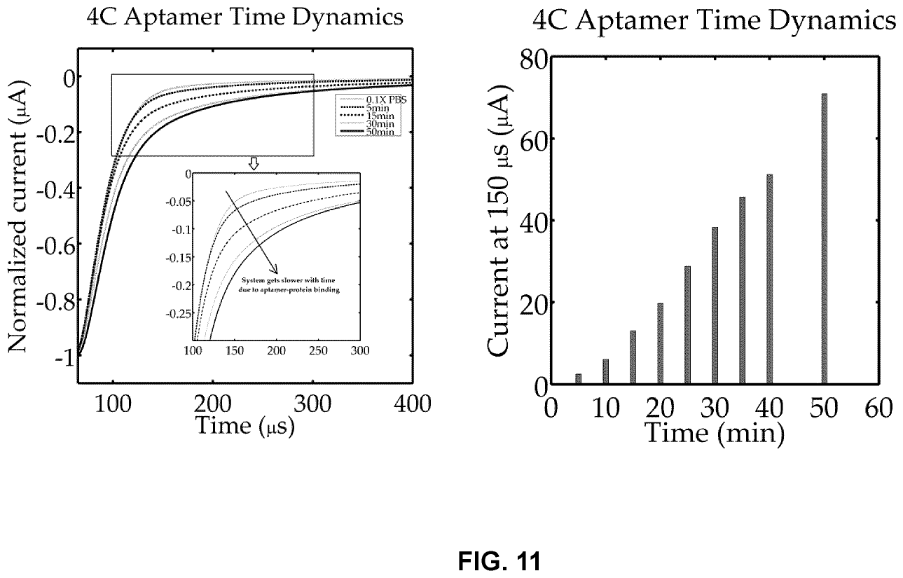

Kinetics of Detection of the SARS-CoV-2 S Protein using an Aptamer System (FIG. 11)

The time dynamics (real-time) measurement of 100 μL of S protein at 1 ng/mL were conducted. Experimental steps are similar as above for FIG. 9. The aptamer-based sensor is also able to get distinguish signal change in 5 minutes of incubation.

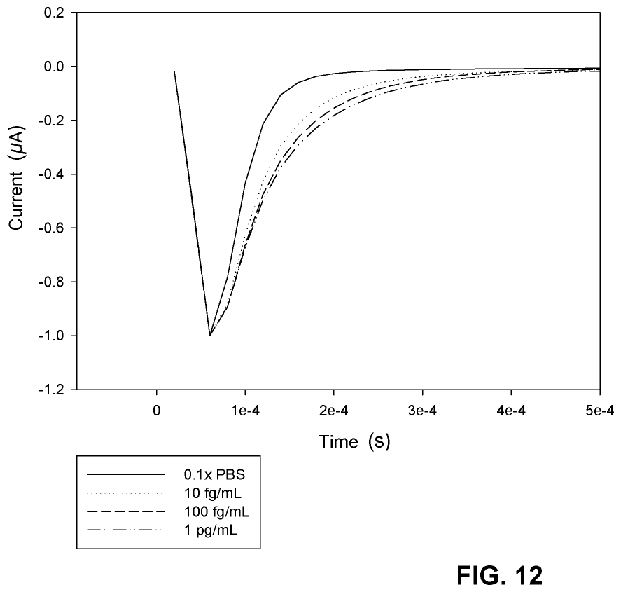

### Measuring the Limit of Detection of the Aptamer-Based Sensor (FIG. 12)

After reading the chips in unbound state (black line) in 0.1×PBS, 100 μL of 10 fg/mL the S protein sample was added to the chip. After 50 minutes of incubation, signal was read out (dotted line). After 3 times washing with PBS, 100 μL of 100 fg/mL of the protein was added to the same chip and incubated for 50 minutes. After incubation, signal was measured (dashed line). Similarly, readout with other concentrations were measured. The sensor was able to detect samples as low as 100 fg/m L.

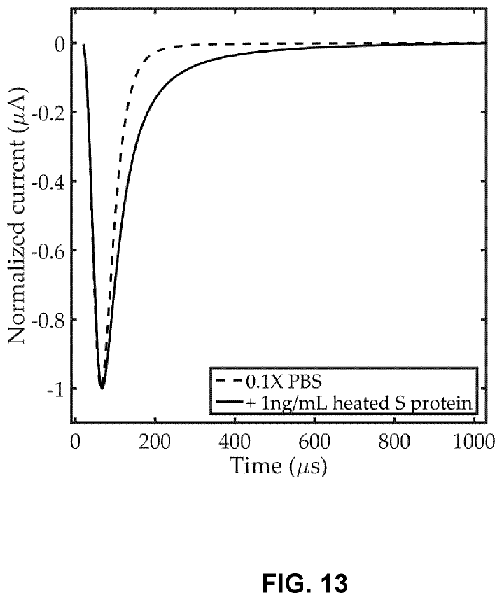

### Detection of Heat-Treated Spike Protein (FIG. 13)

Heat treated spike protein (following standard heat treatment protocol used for inactivation of CoV-2 virus which is 30 minutes incubation at 65° C.) was used to evaluate the performance of the system. After reading the unbound state of the chip, the chip was incubated under heat-treated S protein. Heat-treatment does not have an adverse effect on systems detection capability. This means that the system can be translatable to any sort of heat inactivated virus. Heat treatment process can be added to the prototype device to lower the cross-contamination chance between users.

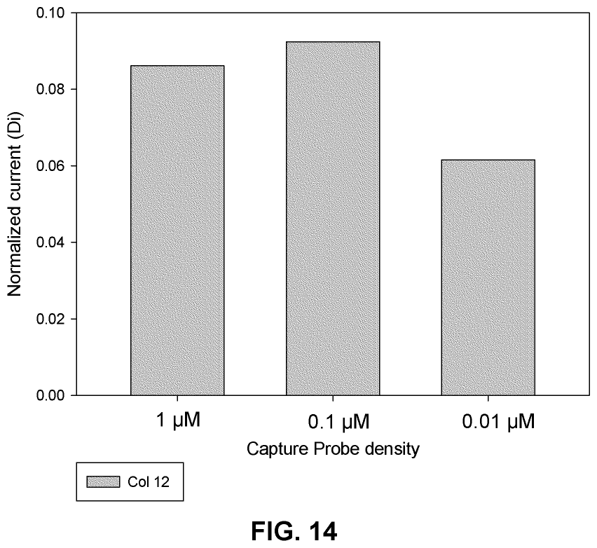

### Capture Probe Density on Detection of Viral Particles (FIG. 14)

Optimization experiments were performed to fine tune the system for the detection of viral particles. In this experiment, the initial iMPs' density immobilized on gold surface was changed. 1 μM dsDNA (P1/P2) concentration is a standard protocol that is used for protein detection. The performance of the sensor with lower concentration of probe (0.1 μM and 0.01 μM) was compared, considering that viral particle is significantly bigger than protein, which may require a more space to reach the surface.

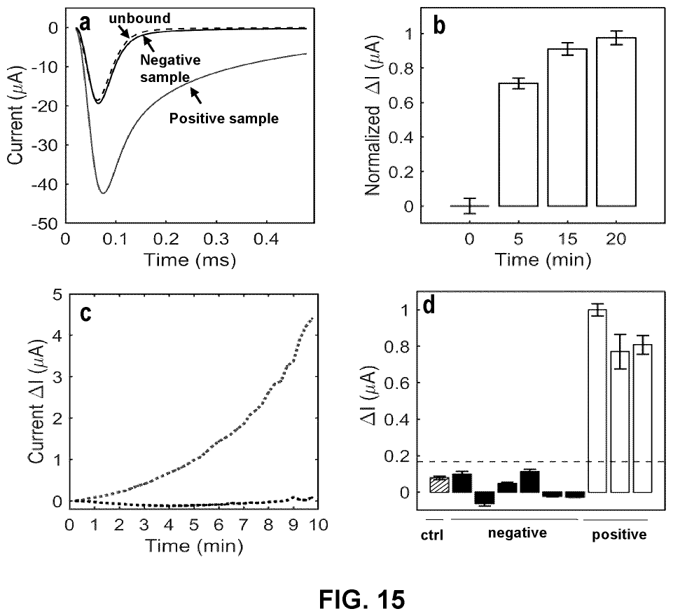

### Detection of SARS-CoV-2 in Human COVID-19 Patient Samples (FIG. 15)

Analysis of human COVID-19 patient samples (FIG. 15a) include SARS-CoV-2 positive saliva sample (bottom solid line), a SARS-CoV-2 negative saliva sample (top solid line) and healthy human saliva (dashed lines). Chronoamperometry traces were collected with a potential step of +500 mV. As seen in FIG. 15b, a rapid response of the sensors upon introduction of patient saliva is observed, as demonstrated with the significant signal increase within 5 minutes. A positive patient sample was incubated with the sensors and the response of the system was monitored (see FIG. 15c, curve trending upwards). The signal differentiated from negative control of healthy donor saliva (FIG. 15c bottom curve, no significant upward increase) in the first 2 minutes. FIG. 15d represents the result of a blinded patient saliva sample analysis. Sensors were challenged with saliva samples collected from SARS-CoV-2 positive and SARS-CoV-2 negative patients. The threshold line indicates mean current +3 times standard deviations of signals collected from sensors acting as negative controls. If the current change for any sample was higher than the threshold, the samples was considered as SARS-CoV-2 positive. Error bars represent standard deviations. At least 3 individual measurements were performed for each sample.

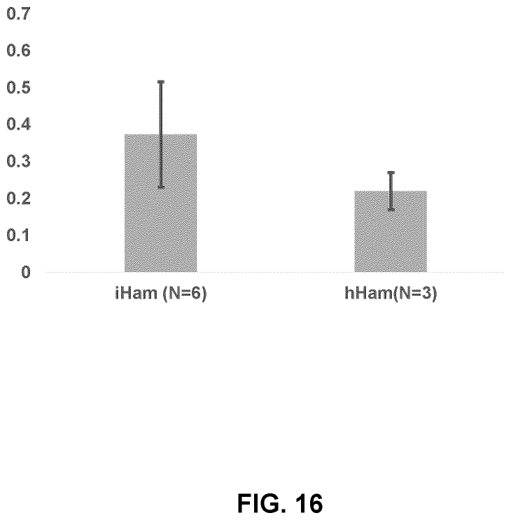

Detection of SARS-CoV-2 in COVID-19 Infected Hamsters for in vivo Demonstration of Sensors (FIG. 16)

A handheld detection device prototype substantially similar to the one presented in FIGS. 18 and 19 was used. The only difference with the device of FIGS. 18 and 19 was that the device, in this example, used an open electrode and was not provided with a capillary saliva collection tip and that it used a printed circuit board (PCB) to take measurements. The device was not provided with a screen but was connected to a computer to transfer data. The handheld detection device prototype was sent to Cl3+ facility and their staff were trained to use the sensors for in vivo study of COVID-19 in infected hamsters (iHam) vs. healthy hamsters (hHam). Currents are normalized.

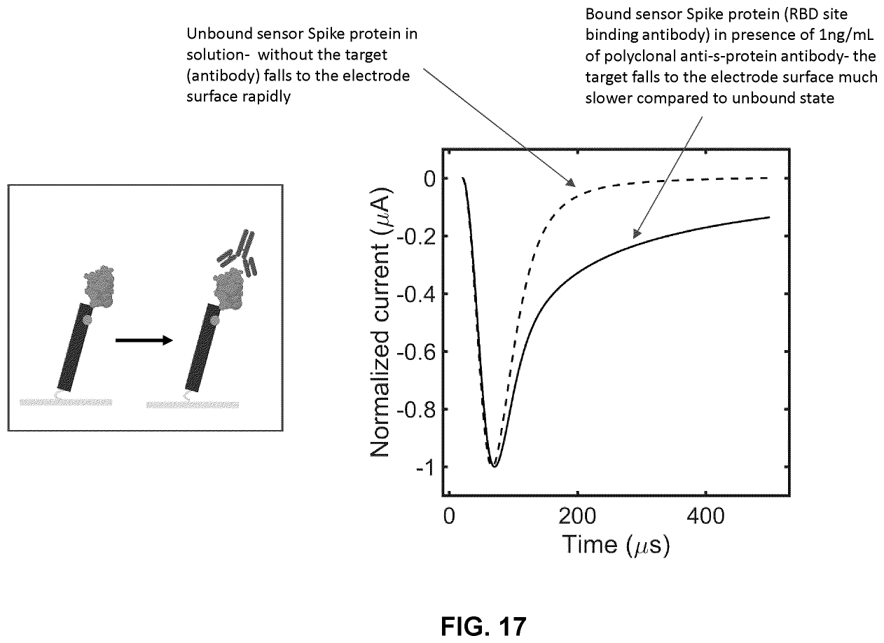

### Detection of Circulating Antibodies (FIG. 17)

The same concept of sensor was used to build another sensor that can detect antibody in bodily fluids. In this case, a P2 probe-conjugated with spike protein was used keeping the P1 probe as same as before. The sensor was used to detect anti-S-protein antibodies (polyclonal anti-S antibody at concentration of 1 ng/mL, 100 μL sample). After initial readout for unbound state (dashed line), the antibody was added to the chip and incubated for 50 minutes. After incubation, the current decay was measured by CA (solid line).

### Handheld Device for Saliva Sampling and Electrochemical Analysis (FIG. 18)

A handheld device can be constructed, which houses the electronics components needed to take electrochemical measurements (FIG. 18). The internal components of the handheld device can include a potentiostat, microcontroller/microprocessor, battery, and connections for an LCD and buttons for the external panel. The external components will consist of the LCD, a socket for charging the device, a socket to connect the cable which interfaces with the disposable device, and buttons for user input, including an on/off switch, a button to begin the electrochemical measurement, and a reset button to take a new measurement. The handheld device housing itself can be 3D printed for prototyping, machined or cast using conventional manufacturing processes. Each electrode from the handheld device has a wired connection to the potentiostat, which takes instructions from the microcontroller/microprocessor. Any commercially available miniature potentiostat solution can be used to enable the handheld device but should exist as an integrated circuit or system on a chip, and have microamp sensitivity and microsecond sampling resolution. Possible embodiments include using the ARM-based ADuCM355 (Analog Devices, Inc), a system-on-a-chip, which integrates the potentiostat and microcontroller, or the LMP91000 (Texas Instruments), an integrated circuit potentiostat. Depending on the embodiment, additional components such as a microcontroller/microprocessor, amplifiers and filters, sensors, serial interface circuitry, and standard electrical components can be further used. All components can be powered by any simple power supply, in practice at 5V and 1A, and no more than 10V and 2A, and can be housed within the handheld device. The device can be passively cooled without fans or other mechanical points of failure. The entire device can be in a handheld format, and during use, only a single cable can connect it to the disposable device. A second cable could connect the handheld device to an electrical outlet for battery charging. For measurements to be taken, the operator will first attach the cable and disposable device to the handheld device and turn the device on with a switch using the external panel. The operator will place the saliva collection tip of the disposable device into the patient's mouth, exposing the tip and its capillary channels to saliva. The operator can start a measurement by pressing a button on the external panel, and the integrated potentiostat/microcontroller solution will take chronoamperometric measurements from each of the three working electrodes for the positive, negative, and experimental conditions. These signals can then be processed to determine whether the patient saliva has SARS-CoV-2 present. The result can then be presented to the operator and patient on the LCD. All electronic components can be integrated with a custom printed circuit board, and all software written and deployed using any available environment compatible with the potentiostat and microcontroller/microprocessor used.

### Disposable Device for Sample Collection and Electrochemical Analysis (FIGS. 18 and 19

The disposable device can include a sensing component, passive saliva collector, and adapter to the handheld device. The sensing component can be made up of wires of substantially equal diameter, with possible embodiments existing as wires of a diameter between 0.1 to 1 millimeter, including gold wire for the three working electrodes, platinum wire for the counter electrode, and silver wire for the reference electrode. Each wire can either be solid gold, platinum, or silver metal or as a plating/coating on an inexpensive metal. All five microwires can be spaced apart and positioned within a 3D printed cylindrical mould with a 0.5 cm inner diameter. To fabricate the disposable sensor, the wire bundle is suspended in a mould in which polydimethylsiloxane (PDMS) is poured into to cure. The electrode wires are extended by 1 cm at the back end of the device to allow for electronic connection. To ensure that the electrode active area is flush with the PDMS, a cut perpendicular to the device is made to expose the electrodes. At the surface of these electrodes, the DNA-based reagentless sensors can be deposited (FIG. 18—inset). The PDMS enclosure ensures that the wires are electrically separated and prevents saliva from moving up the length of the wire. The passive saliva collector can be 3D printed to fit snuggly over the entire sensing component and can include a nozzle and sampling reservoir near the tip. The nozzle can either be patterned with microfluidic channels and treated with a hydrophilic coating, or filled with silica capillary tubes, in order to initiate passive capillary fluid flow once the nozzle comes in contact with saliva. This action will cause the saliva to move towards and fill the 50 μL sampling reservoir, at which point the saliva will contact the sensing component (FIG. 19b). Positive and negative controls can be determined within the same sensor from measuring at different working electrodes (FIG. 19a). The back end of the sensing component can be closed off by a 3D printed adapter which adapts the five microwire connections to a plug and cable which can be connected to the handheld device (FIG. 18). The adapter can have holes to feed through the microwire ends to the larger metal terminals of the plug and fixed electrically using solder cup or crimp connections. The adapter and passive saliva collector can also be screw threaded such that they can lock together and enclose the sensing component. All 3D printed components can be designed with any CAD software (such as Autodesk Fusion, Solidworks, etc.) and printed with <100 μm resolution using a high-resolution 3D printer (such as the MiiCraft 100 series printer).

### Examples 3 to 12

In the following Examples 3 to 12, unless mentioned otherwise, the sensors were including IMPs having the same linker (24mer dsDNA) and redox reporter (ferrocene). The iMPs were immobilized on a gold nanostructured microelectrode as mentioned above. Only the receptor was modified for each application as specified in the following Examples.

### Example 3

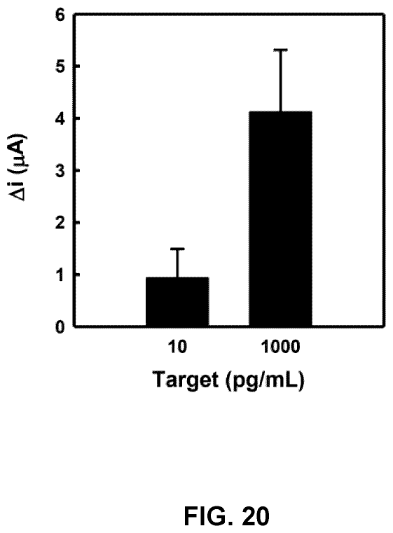

### Long Term Stability of iMPs (FIG. 20)

Previously fabricated troponin I-binding iMP sensors (receptor: troponin I-specific antibody) were stored at 4° C. for 8 months. After the long-term storage, the sensors were used to detect target troponin I proteins with concentrations 10 pg/mL and 1 ng/mL in saliva sample. The signal change correlated with previously reported data indicating that sensors are stable.

### Example 4

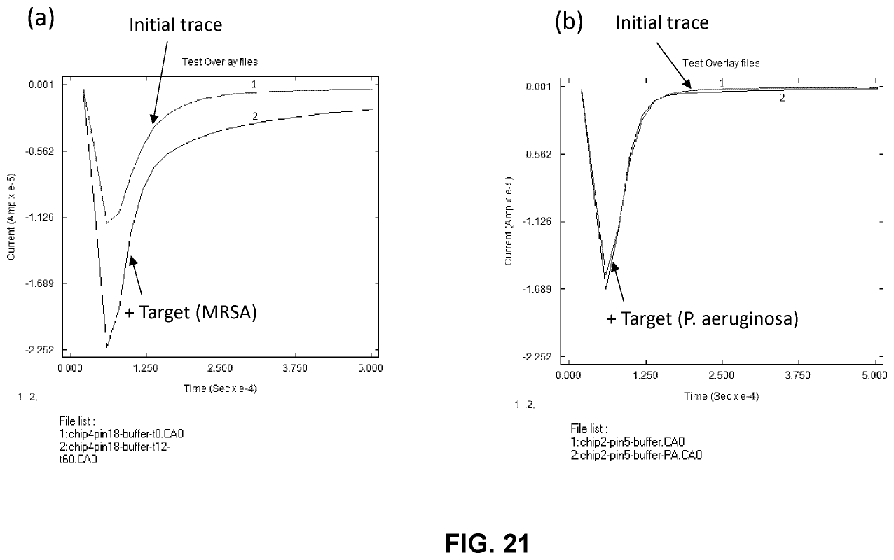

### Detection of Bacteria with iMPs (FIG. 21)

MRSA-binding iMP sensors (receptor: MRSA-specific antibody) were used for detection of bacteria. 500 CFU/mL of target bacteria, Methicillin-resistant Staphylococcus aureus (MRSA) was added and a signal change was observed for bound probes after 60 minutes (FIG. 21a). Negative control bacteria, 500 CFU/mL of Pseudomonas aeruginosa did not show significant change in the unbound vs. bound state (FIG. 21b).

### Example 5

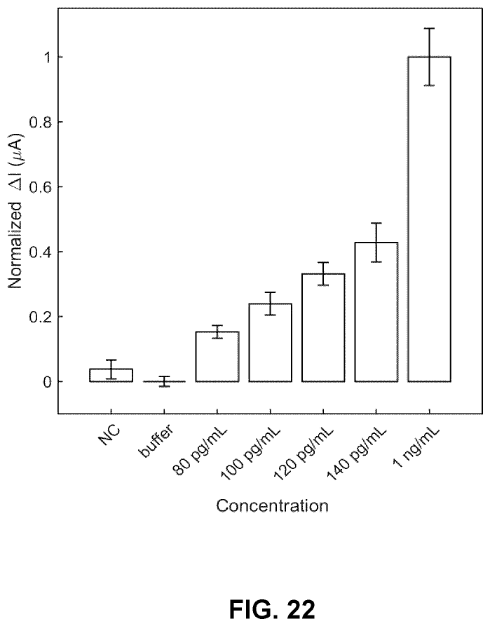

### Analytical Resolution of the Sensor in the Clinically Relevant Range of BNP (FIG. 22)

BNP-binding iMP sensors were constructed using the standard protocol with NMEs on the silicon wafers, and with an antibody specific to the BNP as the receptor. Sensors were incubated with 80, 100, 120, 140 pg/mL, and 1 ng/mL BNP proteins in 0.1×PBS solution. Negative control (NC) sensor was prepared using same protocol, but the antibody specific to BNP was replaced with BSA, and the sensor was incubated with 1 ng/mL BNP protein in 0.1×PBS. The data shows statistically significant changes in current values for a concentration difference of only 20 pg/mL of BNP protein, and the NC sensor did not show any significant current change even with 1 ng/mL of target protein.

### Example 6

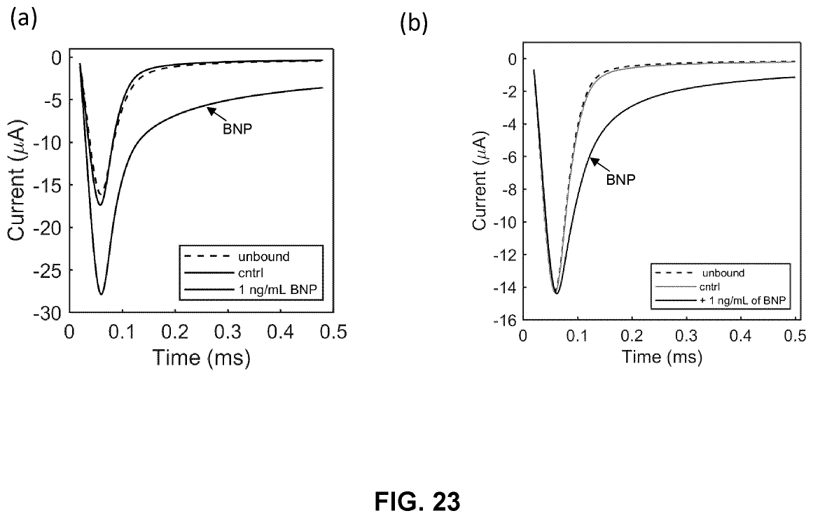

### Detection of BNP in Different Body Fluids (FIG. 23)

BNP-binding iMP sensors were constructed using the standard protocol with NMEs on the silicon wafers, and with an antibody specific to the BNP as the receptor. The sensors were incubated with 1 ng/mL BNP proteins in human saliva (FIG. 23a) and whole human blood (FIG. 23b), each for 50 mins. The control sensors were prepared using the same protocol and were incubated in saliva (FIG. 23a) or whole human blood (FIG. 23b) for 50 mins without target spiked in the fluids.

### Example 7

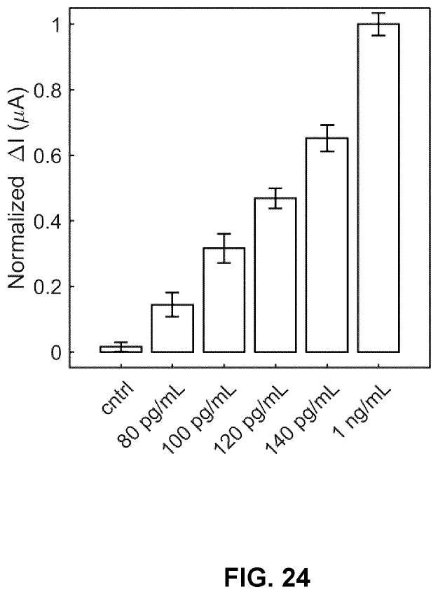

### Analytical Resolution of the Sensor in the Clinically Relevant Range of BNP in Human Blood (FIG. 24)

BNP-binding iMP sensors were constructed using the standard protocol with NMEs on the silicon wafers, and with an antibody specific to the BNP as receptor. Sensors were incubated with 80, 100, 120, 140 pg/mL, and 1 ng/mL BNP proteins spiked in whole human peripheral blood. The control sensor was prepared using same protocol and the sensor was incubated in whole blood without spiking it with target BNP protein. The data shows statistically significant changes in current values for a concentration difference of only 20 pg/mL of BNP protein, and the control sensor did not show any significant current change even with 1 hr incubation in the blood.

### Example 8

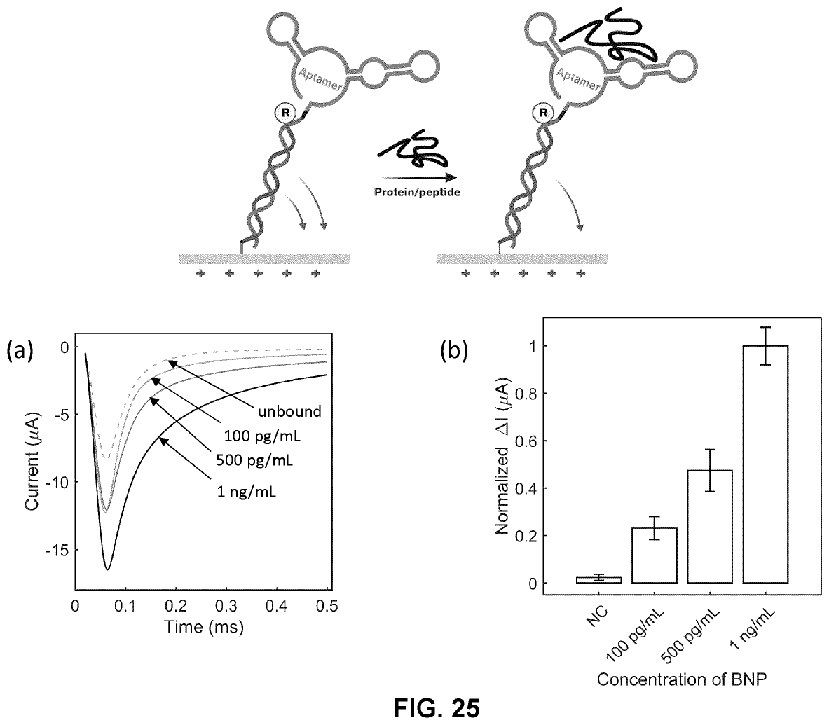

### Detection of BNP with a Molecular Pendulum Aptamer-Based Sensor (FIG. 25)

BNP-binding iMP aptasensors (i.e., iMPs using a BNP-specific aptamer recognition element instead of an antibody receptor) were constructed using the standard protocol with NMEs on the silicon wafers. The sensors were incubated with 100 pg/mL, 500 pg/mL, and 1 ng/mL of BNP proteins in 0.1×PBS. The negative control sensor was prepared using same. FIG. 25a shows the collected choronoamperometric current signals using standard Epsilon device. FIG. 25b shows the concentration-dependent signal change for BNP in buffered solution. The data suggest that the aptasensor can resolve the clinically relevant BNP levels.

### Example 9

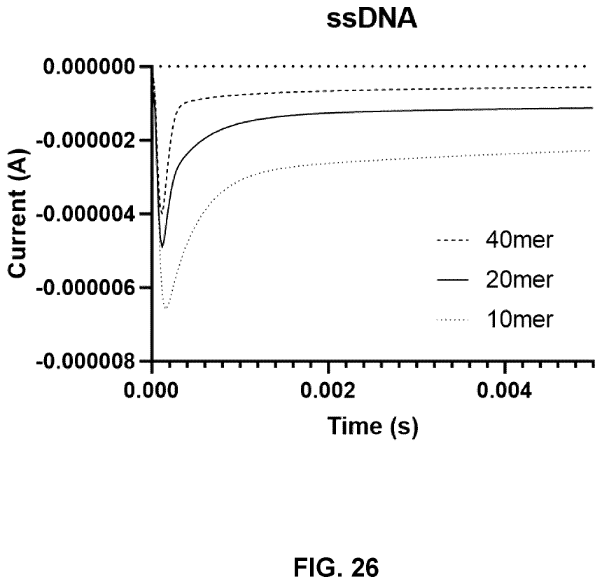

### Length Dependency of ssDNA on Signal Output (FIG. 26)

The faradaic current is dependent on the length of ssDNA used as the linker in the iMP with ferrocene redox reporter and no receptor. The 10 nucleotide ssDNA sequence gave the largest peak current and a 40 nucleotide ssDNA sequence gave the smallest peak current in CA measurements.

### Example 10

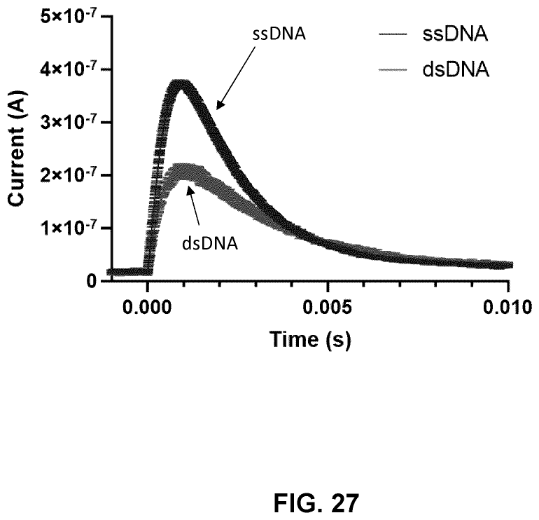

### Chronoamperometric Measurements of ssDNA Versus dsDNA (FIG. 27)

Comparison of chronoamperometric measurements between 20mer ssDNA and dsDNA (n=5, standard error). The CA signal difference between ssDNA and dsDNA, both with ferrocene redox reporter and no receptor, is significant, where ssDNA has a much steeper slope compared to dsDNA.

### Example 11

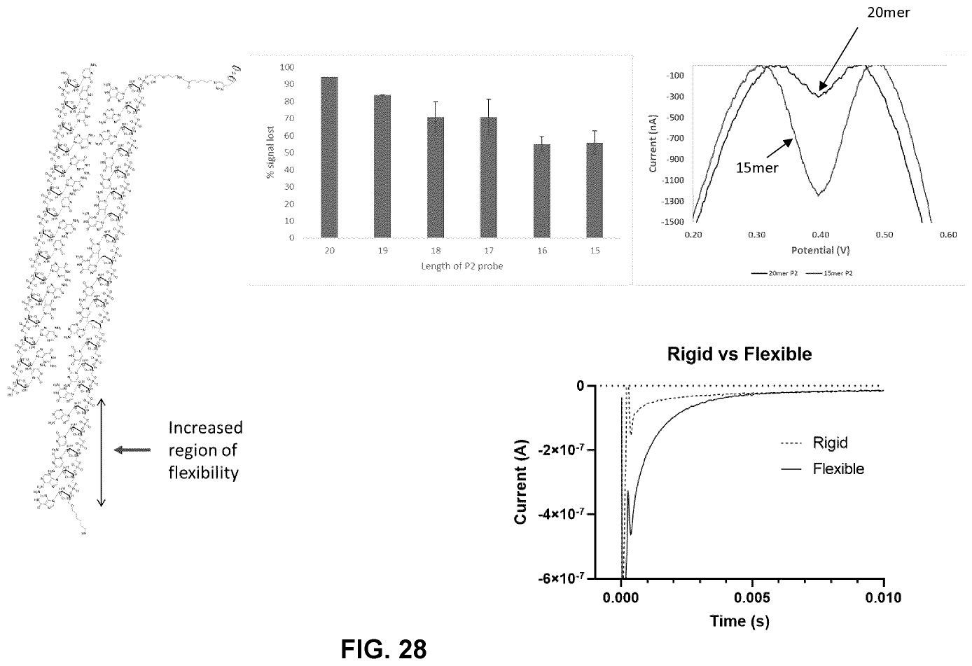

### Introduction of Increased Flexibility on the iMP (FIG. 28)

Increasing the flexibility of the iMP sensor through modulating the base pairing at the 3′ end of P2. The percentage of signal lost decreases as the region of increased flexibility increases. This is further illustrated in a square wave voltammogram comparing a fully complementary, rigid iMP versus a more flexible iMP. CA data shows that the faradaic current is more prominent in a more flexible probe (P2-12) versus the rigid probe (P2-20).

### Example 12

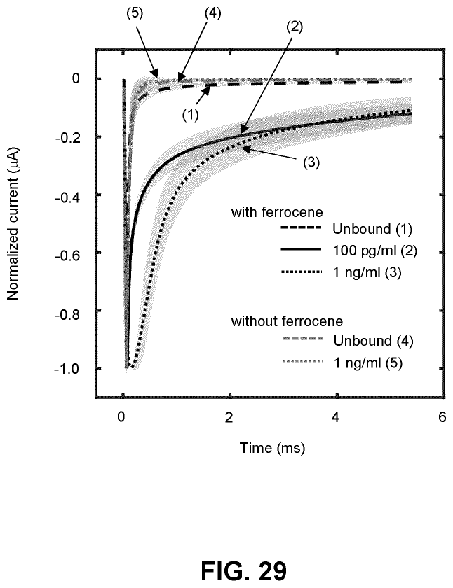

### Capacitive Versus Faradaic Currents for iMP Sensors (FIG. 29)

When dsDNA iMPs lacking ferrocene were tested, both unbound and bound MPs produce only small background capacitive currents. When comparing the same unbound and troponin I bound iMPs with ferrocene attached as redox reporter, the faradaic current responses are significant, indicating the signal produced is faradaic, and indicating the negatively charged dsDNA iMP configuration requires a redox probe active at a positive potential. Standard deviations are shown as grey fill lines, with n=5 for each trace.

### Example 13

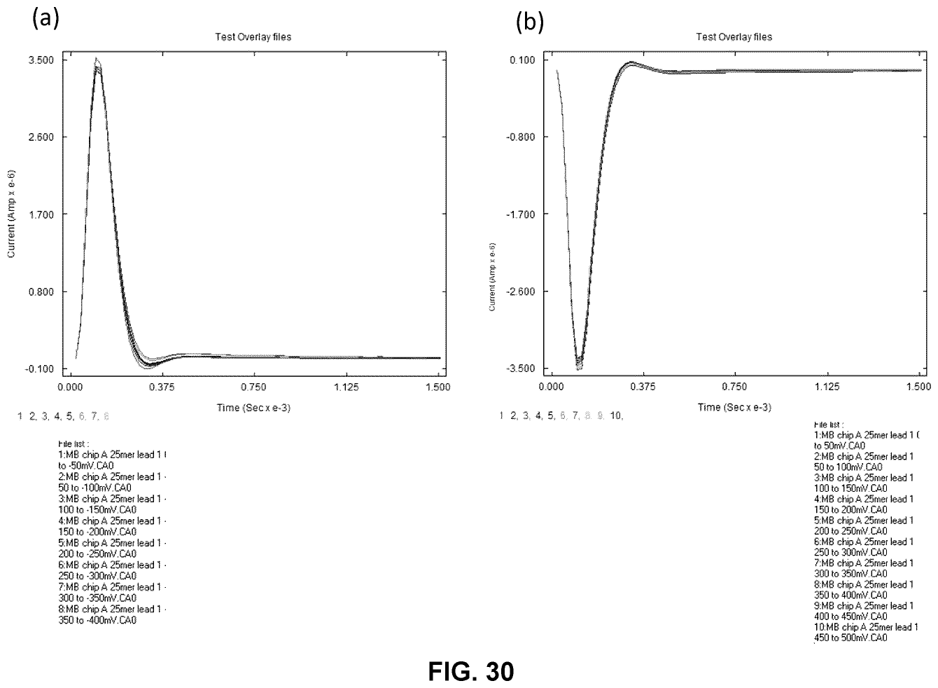

### Verification of Mechanism using Methylene Blue Redox Reporter Molecule on Negatively Charged iMP Sensor (FIG. 30)

A methylene blue reporter molecule, which reduces at a negative potential, was conjugated to a 25mer ssDNA probe and CA steps moving through its reduction potential range were tested (FIG. 30a). No significant change in CA signal was seen at its reduction potential, indicating that the negative reduction potential is not compatible with negatively charged probes. A positive potential range (up to 500mV as the conventional iMP) was next used (FIG. 30b) to rule out capacitive contributions to signal changes, again with no significant changes in CA signal as expected. These results indicate that the iMP operation requires a positive potential reactive redox reporter for a negatively charged iMP (e.g., dsDNA).

1. An electrochemical biosensor comprising a plurality of inverted molecular pendulums (iMPs) and a biosensor electrode, wherein each one of the iMPs comprises
a linker having a first end and a second end, the first end of the linker being bound to a surface of the biosensor electrode,
a receptor for a target analyte, the receptor being bound to the second end of the linker, and
a redox reporter bound to the linker, wherein the redox reporter is reactive at positive potential when the linker presents a net negative charge and the redox reporter is reactive at negative potential when the linker presents a net positive charge,
wherein upon application of an electric field, the biosensor is characterized by
an iMPs unbound state, where no target analyte is bound to the receptor, at which the iMPs are displaced towards the biosensor electrode surface and electron transfer from the iMPs towards the biosensor electrode occurs at an unbound electron transfer rate,
an iMPs bound state, where the target analyte is bound to the receptor, at which the iMPs are displaced towards the biosensor electrode surface and electron transfer from the iMPs towards the biosensor electrode occurs at a bound electron transfer rate.

2. The biosensor of claim 1, wherein upon binding of the target analyte to the receptor at the applied electric field, an electrochemical signal is produced translating a difference between the unbound electron transfer rate and the bound electron transfer rate.

3. The biosensor of claim 1 or 2, wherein upon application of the electric field, the redox reporter causes an electron transfer as the iMPs approach the biosensor electrode surface.

4. The biosensor of any one of claims 1 to 3, wherein the electron transfer rate is dependent on a time rate at which the iMPs are displaced.

5. The biosensor of any one of claims 1 to 4, wherein the unbound electron transfer rate is dependent on a time rate at which the unbound iMPs are displaced.

6. The biosensor of any one of claims 1 to 5, wherein the bound electron transfer rate is dependent on a time rate at which the bound iMPs are displaced.

7. The biosensor of any one of claims 1 to 6, wherein the iMPs displacement towards the biosensor electrode surface substantially corresponds to a tilting movement of the iMPs.

8. The biosensor of any one of claims 1 to 7, wherein upon application of the electric field, the redox reporter touches the biosensor electrode surface and the electron transfer is based on a redox reaction or electron tunneling current.

9. The biosensor of any one of claims 1 to 8, wherein the redox reporter is bound to the linker close to the second end thereof.

10. The biosensor of any one of claims 1 to 9, wherein the redox reporter is covalently bound to the linker.

11. The biosensor of any one of claims 1 to 10, wherein the linker comprises a double-stranded DNA (dsDNA), single-stranded DNA (ssDNA), charged polymers, uncharged polymers, or any combination thereof.

12. The biosensor of any one of claims 1 to 10, wherein the linker is negatively charged and comprises a DNA/DNA duplex, a PNA/DNA duplex, a PNA/PNA duplex where one or both of the PNA are modified with negative charged amino acids, a rigid anionic polyelectrolyte, a rigid negatively charged peptide, or any combination thereof.

13. The biosensor of any one of claims 1 to 12, wherein the redox reporter comprises ferrocene, [Co(GA)₂(phen)] (GA=glycolic acid, phen=1,10-phenathroline), metal nanoparticles (e.g., Au, Pt, Pd, Ag, Cu), pyrroloquinoline quinone (PQQ), benzoquine, Osmium(III) complexes such as Os(bpy)Cl₂³⁺, diphenylamine, or any combination thereof.

14. The biosensor of any one of claims 1 to 13, wherein the redox reporter has a redox state change above 0 mV.

15. The biosensor of any one of claims 1 to 10, wherein the linker is positively charged and comprises a PNA/PNA duplex with lysines, a rigid cationic polyelectrolyte, a rigid positively charged peptide, or any combination thereof.

16. The biosensor of any one of claims 1 to 10 and 15, wherein the redox reporter comprises methylene blue, ruthenium(III) complexes such as Ru(NH₃)₆³⁺, neutral red, toluidine blue, phenosafranine, or any combination thereof.

17. The biosensor of any one of claims 1 to 10, 15 and 16, wherein the redox reporter has a redox state change below 0 mV.

18. The biosensor of any one of claims 1 to 17, wherein the linker has a length ranging from about 5 nm to about 20 nm.

19. The biosensor of any one of claims 1 to 14, wherein the linker comprises a ssDNA having a length ranging from about 10mer to about 100mer.

20. The biosensor of any one of claims 1 to 14, wherein the linker comprises a dsDNA having a length ranging from about 15mer to about 60mer.

21. The biosensor of any one of claims 1 to 20, wherein the linker is rigid along a length thereof.

22. The biosensor of any one of claims 1 to 14 and 20, wherein the linker comprises a dsDNA having a first DNA strand and a second DNA strand, the first DNA strand is bound to the surface of the biosensor electrode at the first end of the linker and the second DNA strand is modified by removing nucleotides from the 3′ end thereof thereby defining an iMPs flexibility region at the first end of the linker.

23. The biosensor of claim 22, wherein the number of removed nucleotides is adjusted such that the difference between the number of nucleotides in the first DNA strand and the number of nucleotides in the second DNA strand is from 1 to 15.

24. The biosensor of claims 22 and 23, wherein the iMPs are rigid in a rigid region comprised between the second end of the linker and the flexibility region.

25. The biosensor of any one of claims 1 to 24, wherein the iMPs form a molecular monolayer at the surface of the biosensor electrode.

26. The biosensor of any one of claims 1 to 25, wherein the receptor comprises an antibody, a nanobody, an antigen, an aptamer, an aptamer fragment, a molecular imprint, a protein receptor, DNA, a microorganism, a protein/enzyme substrate, or any combination thereof.

27. The biosensor of any one of claims 1 to 26, wherein the receptor comprises an antibody, a protein or an aptamer.

28. The biosensor of any one of claims 1 to 26, wherein the receptor comprises an antibody selected from the group consisting of anti-MRSA antibody, anti-MSSA antibody, anti-E. coli antibody, anti-Tuberculosis antibody, anti-pseudomonas aeruginosa antibody, anti-S-protein antibody, anti-troponin I antibody, anti-troponin T antibody, anti-IgE antibody, anti-BNP antibody, anti-BDNF antibody, anti-p53 antibody, anti-AFP antibody, anti-CEA antibody, anti-TRX antibody, anti-IL-8 antibody, and anti-IL-6 antibody.

29. The biosensor of any one of claims 1 to 26, wherein the receptor comprises a protein selected from the group consisting of S-protein, Brain natriuretic peptide (BNP) protein, troponin I protein, troponin T protein, Natural Human IgE protein, Brain-derived neurotrophic factor (BDNF) protein, Thioredoxin (TRX), IL-6 protein, IL-8 protein, Carcino Embryonic Antigen (CEA) protein, alpha 1 Fetoprotein (AFP), and p53 protein.

30. The biosensor of any one of claims 1 to 26, wherein the receptor comprises an aptamer binding the Receptor binding domain (RBD) site of an S-protein or a BNP-specific aptamer.

31. The biosensor of any one of claims 1 to 30, wherein the biosensor electrode comprises a glassy carbon electrode, a carbon nanotube-modified electrode, an indium tin oxide (ITO) electrode, a platinum electrode, a silver electrode, a gold electrode, or a palladium electrode.

32. The biosensor of any one of claims 1 to 31, wherein the biosensor electrode comprises a gold nanostructured microelectrode or a gold wire electrode.

33. A method of detecting a target analyte in a sample comprising:
providing the electrochemical biosensor of any one of claims 1 to 32;
contacting said biosensor with the sample; and
detecting the electrochemical signal, wherein detection of said signal indicates the presence of the target analyte.

34. A method for in situ detection of a target analyte in a biological fluid, comprising:
contacting the electrochemical biosensor of any one of claims 1 to 32 with the biological fluid; and
detecting the electrochemical signal, wherein detection of said signal indicates the presence of the target analyte.

35. Use of the electrochemical biosensor of any one of claims 1 to 32 to detect the presence of a target analyte in a sample.

36. Use of the electrochemical biosensor of any one of claims 1 to 32 to detect the presence of a target analyte in a biological fluid.

37. The method of claim 34 or the use of claim 36, wherein the biological fluid is saliva, blood, urine, tears, sweat or faeces.

38. The biosensor of any one of claims 1 to 32, the method of any one of claim 33, 34 or 37 or the use of any one of claims 35 to 37, wherein the target analyte comprises a small molecule, a macromolecule, a prokaryotic or eukaryotic cell-derived component (e.g., nucleic acid material), a virus, a bacterium, an antibody, a protein, a cellular extract, or any combination thereof.

39. The biosensor of any one of claims 1 to 32, the method of any one of claim 33, 34 or 37 or the use of any one of claims 35 to 37, wherein the target analyte is a protein comprising Brain natriuretic peptide (BNP) protein, troponin I protein, troponin T protein, Natural Human IgE protein, Brain-derived neurotrophic factor (BDNF) protein, Thioredoxin (TRX), IL-6 protein, IL-8 protein, Carcino Embryonic Antigen (CEA) protein, alpha 1 Fetoprotein (AFP), p53 protein, or spike protein (S-protein), and the target analyte is different than the receptor.

40. The biosensor of any one of claims 1 to 32, the method of any one of claim 33, 34 or 37 or the use of any one of claims 35 to 37, wherein the target analyte is an antibody comprising an anti-S-protein antibody, anti-troponin I antibody, anti-troponin T antibody, Anti-IgE antibody, anti-BNP antibody, Anti-BDNF antibody, anti-p53 antibody, anti-AFP antibody, anti-CEA antibody, anti-TRX antibody, anti-IL-8 antibody, or anti-IL-6 antibody, and the target analyte is different than the receptor.

41. The biosensor of any one of claims 1 to 32, the method of any one of claim 33, 34 or 37 or the use of any one of claims 35 to 37, wherein the target analyte is a bacterium comprising Methicillin-resistant Staphylococcus aureus (MRSA), Methicillin-susceptible Staphylococcus aureus (MSSA), E. coli, Tuberculosis (TB) or Pseudomonas Aeruginosa.

42. The biosensor of any one of claims 1 to 32, the method of any one of claim 33, 34 or 37 or the use of any one of claims 35 to 37, wherein the target analyte is a coronavirus.

43. The biosensor of any one of claims 1 to 32, the method of any one of claim 33, 34 or 37 or the use of any one of claims 35 to 37, wherein the target analyte is a SARS-CoV or a MERS-CoV virus, such as the SARS-CoV-2 virus.

44. The biosensor of any one of claims 1 to 32, the method of any one of claim 33, 34 or 37 or the use of any one of claims 35 to 37, wherein the target analyte is an antibody that is specific to a coronavirus.

45. The biosensor of any one of claims 1 to 32, the method of any one of claim 33, 34 or 37 or the use of any one of claims 35 to 37, wherein the target analyte is an antibody that is specific to a SARS-CoV or a MERS-CoV virus, such as the SARS-CoV-2 virus.

46. The biosensor of any one of claims 1 to 14 and 20, wherein the target analyte is the SARS-CoV-2 virus, the linker comprises a double-stranded DNA (dsDNA), the receptor comprises a protein, an aptamer or an antibody specific to the SARS-CoV-2 spike protein and the redox reporter comprises ferrocene.

47. The biosensor of claim 46, wherein the receptor comprises SARS1 polyclonal anti S1 protein antibody, SARS1 S1 protein, Receptor binding domain (RBD) binding IgG, SARS2 polyclonal anti S1 protein antibody, SARS2 Monoclonal anti S protein antibody, SARS2 Polyclonal anti S1 protein antibody, SARS2 S1 protein, or SARS2 S1 protein.

48. The biosensor of claim 46, wherein the receptor comprises an aptamer targeting the Receptor-Binding Domain of the SARS-CoV-2 spike.

49. A disposable device for detecting a target analyte in a sample comprising:
a collector for collecting the sample;
a sensing component comprising the biosensor as defined in any one of claims 1 to 32 and 38 to 48; and
a connector for connecting the sensing component to an electrochemical measurement device;
wherein the collector and the sensing component are designed for allowing contact between the IMPs of the biosensor and the collected sample.

50. The disposable device of claim 49, wherein the collector is designed to at least partially encase the sensing component.

51. The disposable device of claim 49 or 50, wherein the collector comprises a first portion and a second portion, the first portion being able to contain the collected sample and the second portion encasing the sensing component.

52. The disposable device of claim 51, wherein the first portion comprises a nozzle and a sampling reservoir.

53. The disposable device of claim 52, wherein the nozzle comprises microfluidic channels optionally treated with a hydrophilic coating.

54. The disposable device of claim 52, wherein the nozzle comprises silica capillary tubes.

55. The disposable device of any one of claims 49 to 54, wherein the collector and the connector are provided with complementary locking means to secure the sensing component within the device.

56. The disposable device of any one of claims 49 to 55, wherein the sensing component further comprises two working electrodes, a counter electrode and a reference electrode.

57. The disposable device of claim 56, wherein the electrode of the biosensor and the two working electrodes comprise gold.

58. The disposable device of claim 56 or 57, wherein the counter electrode comprises platinum and the reference electrode comprises silver.

59. The disposable device of any one of claims 56 to 58, wherein each electrode is in the form of a wire.

60. The disposable device of claim 59, wherein the sensing component has a cylindrical form and the wires are positioned spaced apart within the sensing component along a length thereof.

61. The disposable device of any one of claims 56 to 60, wherein the electrodes are held in a matrix of a non-conductive material.

62. The disposable device of claim 61, wherein the non-conductive material comprises a silicon resin.

63. The disposable device of any one of claims 59 to 62, wherein the iMPs are bound to a first end of the biosensor electrode wire and the iMPs are exposed to the sample when the first end is in contact with the collected sample.

64. The disposable device of any one of claims 59 to 63, wherein a first end of each electrode wire is in contact with the sample when collected in the collector.

65. The disposable device of any one of claims 59 to 63, wherein a second end of the wires are in contact with the connector.

66. The disposable device of any one of claims 49 to 65, wherein the sample comprises a biological fluid which is saliva, blood, tears, sweat, urine or faeces.

67. The disposable device of any one of claims 49 to 66, wherein the target analyte is the SARS-CoV-2 virus.
